(19) United States
(12) Patent Application Publication
(10) Pub. No.: US 2003/0219717 A1
(43) Pub. Date: Nov. 27, 2003

(54) Fluorophore complementation products

(76) Inventors: Soren Weis Dahl, Rungsted (DK); Bernard Robert Terry, Frederiksberg (DK)

Correspondence Address: BIRCH STEWART KOLASCH & BIRCH, PO BOX 747, FALLS CHURCH, VA, 22040-0747, US

(21) Appl. No.: 10/370,570

(22) Filed: Feb. 24, 2003

Related U.S. Application Data
(63) Continuation of application No. PCT/DK02/00882, filed on Dec. 19, 2002.

(30) Foreign Application Priority Data
Apr. 19, 2002 (DK) 2002-00591
Jun. 13, 2002 (DK) 2002-00896
Jul. 1, 2002 (DK) 2002-01029

(51) Int. Cl. C12Q 1/00; G01N 33/53; G01N 33/567; C07K 14/475

(52) U.S. Cl. 435/4; 435/7.2; 530/399

(57) ABSTRACT

Fluorescence complementation products with intensity levels mimicking the full length intensities are obtained by introduction of improved folding capabilities with a mutation in position 1 preceding the chromophore. This is particularly seen with the yellow variant of Green Fluorescent Protein (GFP). An additive increase is obtained by splitting the GFP between amino acids 172 and 173. Screening for drugs capable of preventing interaction between proteins is performed by selecting the cells with the highest dynamic range through Fluorescence Activated Cell Sorting (FACS), as illustrated with the ability of FK506 to break the rapamycin induced interaction between FRB and FKBP.

### FIELD OF INVENTION

The present invention relates to various split fluorophore complementation products, especially ways to obtain intense systems with Green Fluorescent Protein (GFP).

### BACKGROUND OF THE INVENTION

It has been suggested to use the reassembly of certain enzyme fragments of the complete enzyme as a measure of protein-protein interactions. Johnsson and Varshavsky (Johnsson, N., Varshavsky, A. (1994) Proc. Natl. Acad. Sci. U.S.A. 91, 10340-10344) disclose reassembly of Ubiquitin. This reassembly is detected through the irreversible cleavage of the fusion by Ubiquitin protease and release of a reporter. As opposed to the two-hybrid technique, this technique includes the possibility of monitoring a protein-protein interaction as a function of time, at the natural sites of this interaction in a living cell.

Similar systems are suggested for the reassembly of other proteins including β-galactosidase (Rossi, F., Charlton, C. A., Blau, H. M. (1997) Proc. Natl. Acad. Sci. U.S.A. 94, 8405-8410), dihydrofolate reductase (DHFR, WO98/34120), and β-lactamase (Wehrman, T., Kleaveland, B., Her, J. H., Balint, R. F., Blau, H. M. (2002) Proc. Natl. Acad. Sci. U.S.A. 99, 3469-3474). The basic concept is that by splitting a functional protein in two fragments, the function is lost. The two fragments are transformed or transfected into cells fused in frame to proteins X and Y, respectively. Binding between proteins X and Y will bring the two fragments close together, increasing the local concentration of the complementing fragments, induce folding of these fragments and produce a functional protein with an activity that is similar to that of the non-fragmented protein. If the function is DHFR activity, the cells will survive only if proteins X and Y bind to each other.

Recently, it has been described to use a somewhat similar system for the assisted reassembly and folding of fragments of fluorescent proteins. As the function is fluorescence, the cells will emit light upon excitation only if protein X and protein Y bind to each other thereby assisting complementation.

Ghosh (I. Ghosh, A. D. Hamilton, L. Regan (2000) J. Am. Chem. Soc. 122, 5658-5659, WO01/87919) describes the use of a GFP variant called sg100 (F64L, S65C, Q80R, Y151L, l167T and K238N). This GFP has single fluorescence excitation and emission peaks at 475 nm and 505 nm, respectively (similar to sg25 described by Palm (Palm, G. J., Zdanov, A., Gaitanaris, G. A., Stauber, R., Pavlakis, G. N., Wlodawer, A. (1997) Nat. Struct. Biol. 4, 361-365)).

Functional GFP fragment complementation is accomplished by co-expressing two independent peptides composed of the first 157 N-terminal amino acids of this GFP (NtermGFP) and the remaining 81 C-terminal amino acids (starting form residue 158) of this GFP (CtermGFP) with each of the GFP peptide fragments being fused to interacting leucine zipper peptides that serve to associate the fragments.

Nagai (T. Nagai, A. Sawano, E. S. Park, A. Miyawaki (2001) Proc. Natl. Acad. U.S.A. 98, 3197-3202) tests a yellow fluorescent GFP variant that has the following mutations: S65G, V68L, Q69K, S72A, T203Y. This variant was split between residues N144 and Y145 within the open 129-145 loop region, and the peptides fused to M13 and calmodulin, respectively, for use in a Ca²⁺ assay. However, when the constructs were transfected individually into HeLa cells, the assay was not reliable.

Thus, there is a need for alternative GFP's for use in this technology.

### SUMMARY OF THE INVENTION

The present application discloses that certain GFPs can be reassembled and form a functional fluorescent protein when expressed as two independent proteins halves. For example, when EGFP is expressed in mammalian cells, choosing a split site located in a loop region between the residues that form the beta-sheet structures of the GFP beta-barrel results in intense fluorescence (Example 5 and Example 7). The present application further illustrates that EYFP is also reassembled and, surprisingly, the fluorescence from the reassembled protein is markedly enhanced if it contains the F64L mutation (Example 9).

The reassembly of proteins does not occur, if the two independent proteins halves are fused to non-interacting proteins. But, when brought together, they are reassembled (Example 11).

### DETAILED DISCLOSURE

The non-fluorescent fragments of fluorescent proteins that can be combined to form one functional fluorescent unit are usually produced by splitting the coding nucleotide sequence of one fluorescent protein at an appropriate site and expressing each nucleotide sequence fragment independently. The fluorescent protein fragments may be expressed alone or in fusion with one or more protein fusion partners.

Thus, one aspect of the invention relates to two GFP fragments comprising an N-terminal fragment of GFP, comprising a continuous stretch of amino acids from amino acid number 1 to amino acid number X of GFP, wherein the peptide bond between amino acid number X and amino acid number X+1 is within a loop of GFP, the two GFP fragments also comprise a C-terminal fragment of GFP, comprising a continuous stretch of amino acids from amino acid number X+1 to amino acid number 238 of GFP.

Amino acid 1 is meant to indicate the first amino acid of GFP. Amino acid 238 is meant to indicate the last amino acid of the GFP.

All residues are numbered according to the numbering of wild type A. victoria GFP (GenBank accession no. M62653) and said numbering also applies to equivalent positions in homologous sequences exemplified by alignment of fluorescent protein sequences in Example 1. Thus, when working with truncated GFPs (compared to wild type GFP) or when working with GFPs with additional amino acids, the numbering is relative to the alignment.

Green Fluorescent Protein (GFP) is a 238 amino acid long protein derived from the jellyfish Aequorea Victoria (SEQ ID NO: 1). However, fluorescent proteins have also been isolated from other members of the Coelenterata, such as the red fluorescent protein from Discosoma sp. (Matz, M. V. et al. 1999, Nature Biotechnology 17: 969-973), GFP from Renilla reniformis, GFP from Renilla Muelleri or fluorescent proteins from other animals, fungi or plants. The GFP exists in various modified forms including the blue fluorescent variant of GFP (BFP) disclosed by Heim et al. (Heim, R. et al, 1994, Proc.Natl.Acad.Sci. 91:26, pp 12501-12504) which is a Y66H variant of wild type GFP; the yellow fluorescent variant of GFP (YFP) with the S65G, S72A, and T203Y mutations (WO98/06737); the cyan fluorescent variant of GFP (CFP) with the Y66W colour mutation and optionally the F64L, S65T, N146l, M153T, V163A folding/solubility mutations (Heim, R., Tsien, R. Y. (1996) Curr. Biol. 6, 178-182). The most widely used variant of GFP is EGFP with the F64L and S65T mutations (WO 97/11094 and WO96/23810) and insertion of one valine residue after the first Met. The F64L mutation is the amino acid in position 1 upstream from the chromophore. GFP containing this folding mutation provides an increase in fluorescence intensity when the GFP is expressed in cells at a temperature above about 30° C. (WO 97/11094).

It is known that fluorescence in wild-type GFP is due to the presence of a chromophore, which is generated by cyclisation and oxidation of the SYG at position 65-67 in the predicted primary amino acid sequence and presumably by the same reasoning of the SHG sequence in other GFP analogues at positions 65-67.

The present examples clearly illustrate how the fluorescence intensity from a reassembled protein is enhanced in GFPs containing the F64L mutations as compared to GFPs without this mutation. Thus, it is preferred that the GFP contains the F64L mutation, either by electing a GFP with this mutation (e.g. EGFP) or to introduce this mutation into the GFP of choice (e.g. YFP as illustrated in Example 8).

In the nomenclature of GFP, an “E” is placed in front of the GFP (EGFP, EYFP, ECFP) to indicate that this particular GFP is encoded by a nucleic acid with codon usage optimised for mammalian cells. Most of these proteins also have an extra valine residue inserted after the initial methionine residue, Met¹. This extra valine residue is not considered in the numbering of the residues. Thus, in a preferred embodiment, the GFP of the present invention is selected from the group consisting of EGFP, EYFP, ECFP, dsRed and Renilla GFP.

Some of the examples of the present application, EGFP is used. Thus, in a preferred embodiment of the invention, the GFP is EGFP. However, Example 8 and Example 11 show that EYFP has certain advantages. Thus, in another preferred embodiment of the invention, the GFP is EYFP. It is also shown that EYFP mutated in position 1 preceding the chromophore (E[F64L]YFP) has specific advantages. Thus, in a preferred embodiment the GFP is E[F64L]YFP.

In the present context, the numbering of wild-type GFP (SEQ ID NO: 1) (Chalfie, M., Tu, Y., Euskirchen, G., Ward, W. W., Prasher, D. C. (1994) Science 263, 802-805, this variant of GFP has a histidine residue in position 231) is used. Based on the crystal structure of GFP (Yang, F., Moss, L. G., Phillips, G. N. (1996) Nat. Biotech. 14, 1246-1251) FIG. 5, Table 1 and the data presented in the examples, it is evident that a split in almost any loop will be re-assembled following appropriate spatial approximation to the complementation fragments assisted by the interaction of the conjugated proteins. For the purpose of this application the term “loop” shall be understood as a turn or element of irregular secondary structure.

Thus, in one aspect, the invention relates to two GFP fragments as described above,

wherein X is 7, 8, 11 or 12, preferably X is 9 or 10 within the Thr9-Val11 loop; or

wherein X is 21, 22, 25 or 26, preferably X is 23 or 24 within the Asn23-His25 loop; or

wherein X is 36, 37, 40 or 41, preferably X is 38 or 39 within the Thr38-Gly40loop; or

wherein X is 46, 47, 56 or 57, preferably X is between 48 and 55 i.e. X is 48, 49, 50, 51, 52, 53, 54 or 55 within the Cys48-Pro56 loop; or

wherein X is 70, 71, 76 or 77, preferably X is between 72 and 75 i.e. X is 72, 73, 74 or 75 within the Ser72-Asp76 loop; or

wherein X is 79, 80, 83 or 84, preferably X is 81 or 82 within the His81-Phe83 loop; or

wherein X is 86, 87, 90 or 91, preferably X is 88 or 89 within the Met88-Glu90loop; or

wherein X is 99, 100, 103 or 104, preferably X is 101 or 102 within the Lys101-Asp103 loop; or

wherein X is 112, 113, 118 or 119, preferably X is between 114 and 117 i.e. X is 114, 115, 116 or 117 within the Phe114-Thr118 loop; or

wherein X is 126, 127, 145 or 146, preferably X is between 128 and 144 i.e. X is 128, 129, 130, 131, 132, 133, 134, 135, 136, 137, 138, 139, 140, 141, 142, 143, 144 within the Ile 128-Tyr145 loop; or

wherein X is 152, 153, 160 or 161, preferably X is between 154 and 159 i.e. X is 154, 155, 156, 157, 158 or 159 within the Ala154-Gly160 loop; or

wherein X is 169, 170, 175, 176, preferably X is between 171 and 174 i.e. X is 171, 172, 173 or 174 within the Ile171-Ser175 loop; or

wherein X is 186, 187, 197 or 198, preferably X is between 188 and 196 i.e. X is 188, 189, 190, 191, 192, 193, 194, 195 or 196 within the Ile188-Asp197 loop; or

wherein X is 208, 209, 215 or 216, preferably X is between 210 and 214 i.e. X is 210, 211, 212, 213 or 214 within the Asp210-Art215 loop.

TABLE 1
GFP secondary structures, GFP wild type
sequence amino acid numbering. α and β indicate
α-helical and β-sheet secondary structures, respectively.

| Name | Position | |
|---|---|---|
| Helix 1 | Lys3-Thr9 | α1 |
| Sheet 1 | Val11-Asn23 | β1 |
| Sheet 2 | His25-Thr38 | β2 |
| Sheet 3 | Gly40-Cys48 | β3 |
| Helix 2 | Pro56-Ser72 | α2 |
| Helix 3 | Asp76-His81 | α3 |
| Helix 4 | Phe83-Met88 | α4 |
| Sheet 4 | Glu90-Lys101 | β4 |
| Sheet 5 | Asp103-Phe114 | β5 |
| Sheet 6 | Thr118-Ile128 | β6 |
| Sheet 7 | Tyr145-Ala154 | β7 |
| Sheet 8 | Gly160-Ile171 | β8 |
| Sheet 9 | Ser175-Ile188 | β9 |
| Sheet 10 | Asp197-Asp210 | β10 |
| Sheet 11 | Arg215-Gly228 | β11 |

Based on the findings disclosed in the examples, it is concluded that appropriate splitting sites in GFP are located in the loop regions between the residues that form the beta-sheet structures of the GFP beta-barrel. Accordingly, splits in GFP are preferably made in the Asn23-His25 loop, the Thr38-Gly40 loop, the Lys101-Asp102 loop, the Phe114-Thr118 loop, the Ile128-Tyr145 loop, the Ala154-Gly160 loop, the Ile171-Ser175 loop, the Ile188-Asp197 loop or the Asp210-Arg215 loop (Table 1, FIG. 5).

The data in the present examples illustrates clearly that the Ala154-Gly160 loop is very well suited for GFP reassembly. This is particularly the case when the GFP is divided between amino acids Q157 and K158 (that is, when X is 157). Thus, a preferred embodiment of the invention relates to two GFP fragments, wherein X is 157 within the Ala154-Gly160 loop.

The data in the present examples also illustrate that the Ile171-Ser175 loop is very useful for GFP reassembly. This is particularly the case, when the GFP is divided between amino acids E172 and D173 (that is, when X is 172). Thus, a preferred embodiment of the invention relates to two GFP fragments, wherein X is 172 within the Ile171-Ser175 loop.

As illustrated in Example 9, fragments having overlapping sequences have certain advantages. Thus one aspect of the invention relates to two GFP fragments comprising

(a) an N-terminal fragment of GFP, comprising a continuous stretch of amino acids from amino acid number 1 to amino acid number X of GFP, wherein the peptide bond between amino acid number X and amino acid number X+1 is within a loop of GFP and

(b) a C-terminal fragment of GFP, comprising a continuous stretch of amino acids from amino acid number Y+1 to amino acid number 238 of GFP, wherein Y<X creating an overlap of the two GFP fragments, and wherein the peptide bond between amino acid Y and amino acid Y+1 is within a loop of GFP.

These overlapping GFP fragments are very attractive in e.g. functional cloning systems where highly flexible linkers sequences are required due to the very diverse nature of the structures of fusion partners. The overlapping fragments permit either of the fusion partners to have a long linker sequence.

For the purposes of deciding the nature of the Y in the C-terminal fragment of GFP defined above, the same considerations as discussed for the value of X applies.

In one embodiment of the invention the overlap is just a few amino acid residues, e.g. X−Y=1, X−Y=2, X−Y=3, X−Y=4, X−Y=5, X−Y=6, X−Y=7, X−Y=8, X−Y=9 or X−Y=10.

Due to the folding characteristics of the folding of GFP, a preferred embodiment of the invention relates to overlapping N-terminal and C-terminal fragments of GFP wherein the peptide bond between amino acid Y and amino acid Y+1 and the peptide bond between amino acid X and amino X+1 is within a loop of GFP. The thereby obtained overlap is an entire α-helix or β-sheet secondary structure

In order to obtain reassembly of the two halves of GFP, it is preferred to have the two halves of GFP fused to interaction partners that will bring said two halves of GFP so close together that the protein halves will fold and form functional GFP. Thus, a preferred embodiment of the invention relates to a fusion protein comprising an N-terminal fragment of GFP as described above conjugated to a first protein of interest. In a particular embodiment the nucleic acid encoding the N-terminal fragment of GFP is fused in frame to the first protein of interest. In similar embodiments, the present invention relates to two GFP fragments as described above, wherein the C-terminal fragment of GFP is conjugated to a second protein of interest. In a particular embodiment, the nucleic acid encoding the C-terminal fragment of GFP is fused in frame to the second protein of interest.

As will be evident to the skilled person, the protein of interest is conjugated to the GFP fragment in the N-terminal or in the C-terminal. However, as illustrated in the examples, conjugation of the first protein of interest to the N-terminal fragment of GFP shall preferably be to the C-terminal of the N-terminal fragment of GFP. Likewise, conjugation of the second protein of interest to the C-terminal fragment of GFP shall preferably be to the N-terminal of the C-terminal fragment of GFP.

As will be evident from the present examples the protein of interest is a protein, a peptide or a non-proteinaceous partner.

In a typical embodiment of the invention, the conjugated protein as described above, wherein the fragment of GFP is conjugated to a protein of interest, further comprises a linker sequence between either fragment of GFP and the corresponding protein of interest.

The linker must be chosen dependent on the protein of interest conjugated to the fragment of GFP. Thus the linker must be flexible. A long linker prevent steric hindrance of the complementation due to the protein of interest. However short linkers keeps the fragments of GFP closer to each other and gives better associations.

The present invention also relates to the N-terminal fragment of GFP as described above. In a similar embodiment, the invention relates to the C-terminal fragment of GFP as described above.

A preferred embodiment of the invention relates to a nucleic acid encoding any of the fragments or fusions proteins described above. In one embodiment, the nucleic acid construct encoding any of the proteins according to the invention described above is a DNA construct. In another embodiment, the nucleic acid construct encoding any of the proteins according to the invention described above is a RNA construct.

One aspect of the invention relates to a cell containing the two GFP fragments described above. In similar embodiments, the invention relates to a cell containing the N-terminal fragment of GFP described above. In similar embodiments, the invention relates to a cell containing the C-terminal fragment of GFP described above.

Numerous cell systems for transfection exist. A few examples of mammalian cells isolated directly from tissues or organs taken from healthy or diseased animals (primary cells), or transformed mammalian cells capable of indefinite replication under cell culture conditions (cell lines). The term “mammalian cell” is intended to indicate any living cell of mammalian origin. The cell may be an established cell line, many of which are available from The American Type Culture Collection (ATCC, Virginia, USA) or similar Cell Culture Collections. The cell may be a primary cell with a limited life span derived from a mammalian tissue, including tissues derived from a transgenic animal, or a newly established immortal cell line derived from a mammalian tissue including transgenic tissues, or a hybrid cell or cell line derived by fusing different cell types of mammalian origin e.g. hybridoma cell lines. The cells may optionally express one or more non-native gene products, e.g. receptors, enzymes, enzyme substrates, prior to or in addition to the fluorescent probe. Preferred cell lines include, but are not limited to, those of fibroblast origin, e.g. BHK, CHO, BALB, NIH-3T3 or of endothelial origin, e.g. HUVEC, BAE (bovine artery endothelial), CPAE (cow pulmonary artery endothelial), HLMVEC (human lung micro vascular endothelial cells), or of airway epithelial origin, e.g. BEAS-2B, or of pancreatic origin, e.g. RIN, INS-1, MIN6, bTC3, aTC6, bTC6, HIT, or of hematopoietic origin, e.g. primary isolated human monocytes, macrophages, neutrophils, basophils, eosinophils and lymphocyte populations, AML-14, AML-193, HL-60, RBL-1, U937, RAW, JAWS, or of adipocyte origin, e.g. 3T3-L1, human pre-adipocytes, or of neuroendocrine origin, e.g. AtT20, PC12, GH3, muscle origin, e.g. SKMC, A10, C2C12, renal origin, e.g. HEK 293, LLC-PK1, or of neuronal origin, e.g. SK-N-DZ, SK-N-BE(2), HCN-1A, NT2/D1.

The examples of the present invention are based on CHO cells. Therefore, fibroblast derived cell lines such as BALB, NIH-3T3 and BHK cells are preferred.

It is preferred that the heterologous conjugates are introduced into the cell as plasmids, e.g. individual plasmids mixed upon application to cells with a suitable transfection agent such as FuGENE so that transfected cells express and integrate all heterologous conjugates (or GFP fragments) simultaneously. Plasmids coding for each conjugate will contain a different genetic resistance marker to allow selection of cells expressing those conjugates. It is also preferred that each of the conjugates also contains a distinct amino acid sequence, such as the HA or myc or Flag markers, that may be detected immunocytochemically so that the expression of these conjugates in cells may be readily confirmed. Many other means for introduction of one or both of the conjugates are evenly feasible e.g. electroporation, calcium phosphate precipitate, microinjection, adenovirus and retroviral methods, bicistronic plasmids encoding both conjugates etc.

Throughout the present invention, the term “protein” should have the general meaning. That includes not only a translated protein, a peptide or a protein fragment, but also chemically synthesized proteins. For proteins translated within the cell, the naturally, or induced, post-translational modifications such as glycosylation and lipidation are expected to occur and those products are still considered proteins.

The term “intracellular protein interaction” has the general meaning of an interaction between two proteins, as described above, within the same cell. The interaction is due to covalent and/or non-covalent forces between the protein components, most usually between one or more regions or domains on each protein whose physico-chemical properties allow for a more or less specific recognition and subsequent interaction between the two protein components involved. In a preferred embodiment, the intracellular interaction is a protein-protein binding.

The recording of the fluorescence will vary according to the purpose of the method in question. In one embodiment the emitted light is measured with various apparatus known to the person skilled in the art. Typically, such apparatus comprises the following components: (a) a light source, (b) a method for selecting the wavelength(s) of light from the source that will excite the luminescence of the luminophore, (c) a device that can rapidly block or pass the excitation light into the rest of the system, (d) a series of optical elements for conveying the excitation light to the specimen, collecting the emitted fluorescence in a spatially resolved fashion, and forming an image from this fluorescence emission (or another type of intensity map relevant to the method of detection and measurement), (e) a bench or stand that holds the container of the cells being measured in a predetermined geometry with respect to the series of optical elements, (f) a detector to record the light intensity, preferably in the form of an image, (g) a computer or electronic system and associated software to acquire and store the recorded information and/or images, and to compute the degree of redistribution from the recorded images.

In one embodiment of the invention, the actual fluorescence measurements are made in a standard type of fluorometer for plates of micro titer type (fluorescence plate reader).

In one embodiment, the optical scanning system is used to illuminate the bottom of a plate of micro titer type so that a time-resolved recording of changes in luminescence or fluorescence can be made from all spatial limitations simultaneously.

In one embodiment, the image is formed and recorded by an optical scanning system.

A variety of instruments exist to measure light intensity. In one embodiment a fluorescence plate reader is used (e.g. Wallac Victor (BD Biosciences), Spectrafluor (Tecan), Flex station (Molecular Devices), Explorer (Acumen)). In another embodiment an imaging plate readers is used (e.g. FLIPR (Molecular Devices) LeadSeaker (Amersham), VIPR (Molecular Devices)). In another embodiment an automated imager is used like Arrayscan (Cellomics), Incell Analyser (Amersham), Opera (Evotec). In a still further embodiment a confocal fluorescence microscope is used (e.g. LSM510 (Zeiss)).

One aspect of the invention relates to a method for detecting the interaction between two proteins of interest comprising the steps of:

(a) providing at least one cell that contains two heterologous conjugates,

the first heterologous conjugate comprising a first protein of interest conjugated to an N-terminal fragment of GFP as described above,

the second heterologous conjugate comprising a second protein of interest conjugated to a C-terminal fragment of GFP as described above; and

(b) measuring the fluorescence from the at least one cell,

fluorescent cells indicating interaction between the two proteins of interest.

In a similar embodiment, the invention relates to a method for monitoring the interaction between two proteins of interest comprising the steps of:

(a) providing at least one cell containing at least one stretch of nucleic acid encoding two heterologous conjugates:

the first heterologous conjugate comprising a first protein of interest conjugated to an N-terminal fragment of GFP as described above,

the second heterologous conjugate comprising a second protein of interest conjugated to a C-terminal fragment of GFP as described above;

(b) culturing the at least one cell under conditions allowing expression; and

(c) measuring the fluorescence from the at least one cell,

fluorescent cells indicating interaction between the two proteins of interest.

In one aspect of the methods, one of the proteins of interest is known, whereas the other protein of interest is an unknown protein. By parallel transfection of the cells with both heterologous conjugates, cells expressing an unknown protein that interacts with the know protein of interest will be fluorescent and thereby easily detectable. In an alternative embodiment of the invention, a cell line is established that stabilly expresses the heterologous conjugate comprising the known protein of interest and a library of heterologous conjugates comprising the potential interaction partners is then transfected into the cells—one per well.

As clearly illustrated in the present examples, the method is useful in detecting compounds that induce interaction between two proteins of interest. Such method comprises the steps of:

(a) providing at least one cell that contains two heterologous conjugates,

the first heterologous conjugate comprising a first protein of interest conjugated to an N-terminal fragment of GFP as described above,

the second heterologous conjugate comprising a second protein of interest conjugated to a C-terminal fragment of GFP as described above; and

(b) measuring the fluorescence from the at least one cell of step (a),

(c) apply a test compound to the at least one cell of step (b)

(d) measuring the fluorescence from the at least one cell of step (c);

an increase in fluorescence observed from step (b) to step (d) indicating that the test compound added in step (c) is capable of inducing interaction between the two proteins of interest.

If a compound that induces interaction between two proteins of interest is known and available, this compound can be useful as a reference compound for the method for detecting compounds that induce interaction between two proteins of interest.

In a case where a compound that induces interaction between two proteins of interest is known, it also opens the possibility to screen for compounds that interfere with a conditional interaction between two protein components. Such method comprises the steps of:

(a) providing at least one cell that contains two heterologous conjugates,

the first heterologous conjugate comprising a first protein of interest conjugated to an N-terminal fragment of GFP as described above,

the second heterologous conjugate comprising a second protein of interest conjugated to a C-terminal fragment of GFP as described above; and

(b) measuring the fluorescence from the at least one cell of step (a),

(c) apply a test compound and the compound that induces interaction between two proteins of interest to the at least one cell of step (b)

(d) measuring the fluorescence from the at least one cell of step (c);

an increase in fluorescence observed from step (b) to step (d) indicating that the test compound added in step (c) does not prevent interaction between the two proteins of interest; whereas an increase in fluorescence observed from step (b) to step (d), which increase is less compared to the increase in fluoresence observed when the test compound is absent and only the compound that induces interaction is present, is indicating that the test compound will interfere with the induced interaction between the two proteins of interest.

One particular advantage of the present method is that it can be carried out in a heterogeneous cell population. This avoids inter alia the steps required to get clonal cells. This is achieved by fluorescence activated cell sorting (FACS) prior to testing. One step in that process is removal of the most green cells, that is the cells wherein t functional fluorescence is achieved even though the two proteins of interest were not supposed to interact. Another step is removal-of the black cells, that is the cells wherein the two heterologous conjugates do not interact e.g. where no or little functional complementation occurs. This could be due to lack of transfection in those cells, a poor expression ratio between the two constructs, or lack of functional expression of either construct. It is presently anticipated that, in both the most green cells and the black cells, the transfection has not taken place as desired, resulting in no, poor, or excessive complementation of the heterologous conjugates. The hereby obtained “medium to low-green” cells are then used in any of the methods described above, or other complementation based methods. The “most green”, “medium green”, “low green” and “black” cells respectively have decreasing levels of fluorescence relative to on another. These levels are predetermined by the skilled artisan in relative proportions

The preferred method for detecting interactions between proteins of interest include an additional FACS. The aim of this second FACS step is to isolate cells with a large dynamic range. The first step is stimulating the “medium to low-green” FACS cells with the compound that induce interaction between two proteins of interest and thereafter allow sufficient time to pass to let the proteins interact and the fluorescent protein fragments fold and become fluorescent. The next step is to subject them to the second FACS step removing the most green cells. The remaining population of cells will have a low to medium background and are still capable of forming the fluorescent protein upon interaction between the two proteins of interest. When the cells have grown to sufficient number, and a number of generations will have diluted the fluorescence, the cells are ready to use in any of the methods outlined above, e.g. detecting compounds that induce interaction between two proteins of interest and to screen for compounds that interfere with a conditional interaction between two protein components.

In a preferred aspect of the methods, the at least one cell is a mammalian cell.

The term “compound” is intended to indicate any sample, that has a biological function or exerts a biological effect in a cellular system. The sample may be a sample of a biological material such as a sample of a body fluid including blood, plasma, saliva, milk, urine, or a microbial or plant extract, an environmental sample containing pollutants including heavy metals or toxins, or it may be a sample containing a compound or mixture of compounds prepared by organic synthesis or genetic techniques. The compound may be small or-ganic compounds or biopolymers, including proteins and peptides.

In another preferred aspect of the methods, the heterologous conjugates are fusion proteins.

This technology has broad applicability. Due to the direct detection of interactions it can be used in genomics and proteomics. The high sensitivity makes it applicable to target discovery and the high specificity makes it applicable to target validation. It can be scaled to Drug Discovery in High Throughput Screening. The technology is quantitative and makes it applicable to nanotechnology and diagnostics.

The invention will be illustrated more specifically in the following non-limiting examples.

### EXAMPLE

### Example 1

Alignment of Fluorescent Proteins

| GenBank entry | Fluorescent protein |
|---|---|
| P42212 | Aequorea victoria green-fluorescent protein |
| AF372525 | Renilla reniformis green fluorescent protein |
| AY015996 | Renilla muelleri green fluorescent protein |
| AY013824 | Aequorea macrodactyla isolate GFPxm |
| AF384683 | Montastraea cavernosa green fluorescent protein |
| AF401282 | Montastraea faveolata green fluorescent protein |
| AY015995 | Ptilosarcus sp. CSG-2001 green fluorescent protein |
| AF322221 | Anemonia sulcata green fluorescent protein asFP499 |
| AF322222 | Anemonia sulcata nonfluorescent red protein asCP562 |
| AF246709 | Anemonia sulcata GFP-like chromoprotein FP595 |
| AF168419 | DsRed Discosoma sp. fluorescent protein FP583 |
| AF168420 | Discosoma striata fluorescent protein FP483 |
| AF168421 | Anemonia majano fluorescent protein FP486 |
| AF168422 | Zoanthus sp. fluorescent protein FP506 |
| AF168423 | Zoanthus sp. fluorescent protein FP538 |
| AF168424 | Clavularia sp. fluorescent protein FP484 |

The alignment is presented in FIG. 16.

### Example 2

Construction of EGFP Complementation Fragment Probes

Anti-parallel leucine zippers (called NZ and CZ) that can bind to each other within prokaryotic and eukaryotic were fused to different fragments of GFP to evaluate the optimal site for splitting GFP for use of such fragments in molecular complementation experiments, including bimolecular fluorescence complementation experiments. NZ and CZ leucine zippers were prepared by annealing and ligating phosphorylated oligo nucleotides 2110-2115 (for NZ zipper, see Table 2) or phosphorylated oligo nucleotides 2116-2121 (for CZ zipper), into Ncol-BamHI cut pTrcHis-A vector (commercially available from Invitrogen) producing vector PS1515 (expression vector encoding NZ zipper) or PS1516 (expression vector encoding CZ zipper). The oligos ligated in NZ and CZ annealing mixes 1 produced the coding sequences of the N-terminal parts of the NZ and CZ zippers. The oligos ligated in NZ and CZ annealing mixes 2 produced the coding sequences of the middle parts of the NZ and CZ zippers and the oligos ligated in NZ and CZ annealing mixes 3 produced the coding sequences of the C-terminal parts of the NZ and CZ zippers.

Annealing Primer Pairs for NZ Zipper

| | |
|---|---|
| NZ annealing mix 1 | |
| Forward oligo 2110 (1 μM) | 5 μl |
| Reverse oligo 2111 (1 μM) | 5 μl |
| 50 mM Tris-HCl, 10 mM MgCl₂, pH 8.0 | 2 μl |
| H₂O | 8 μl |
| NZ annealing mix 2 | |
| Forward oligo 2112 (1 μM) | 5 μl |
| Reverse oligo 2113 (1 μM) | 5 μl |
| 50 mM Tris-HCl, 10 mM MgCl₂, pH 8.0 | 2 μl |
| H₂O | 8 μl |
| NZ annealing mix 3 | |
| Forward oligo 2114 (1 μM) | 5 μl |
| Reverse oligo 2115 (1 μM) | 5 μl |
| 50 mM Tris-HCl, 10 mM MgCl₂, pH 8.0 | 2 μl |
| H₂O | 8 μl |

Each of the annealing mixes were heated at 80° C. for 2 minutes on a pre-heated Hybaid OmniGene PCR machine which was subsequently turned off and allowed to cool to room temperature (about 10 min). The fragments were subsequently put on ice.

Annealing Primer Pairs for CZ Zipper

| | |
|---|---|
| CZ annealing mix 1 | |
| Forward oligo 2116 (1 μM) | 5 μl |
| Reverse oligo 2117 (1 μM) | 5 μl |
| 50 mM Tris-HCl, 10 mM MgCl₂, pH 8.0 | 2 μl |
| H₂O | 8 μl |
| CZ annealing mix 2 | |
| Forward oligo 2118 (1 μM) | 5 μl |
| Reverse oligo 2119 (1 μM) | 5 μl |
| 50 mM Tris-HCl, 10 mM MgCl₂, pH 8.0 | 2 μl |
| H₂O | 8 μl |
| CZ annealing mix 3 | |
| Forward oligo 2120 (1 μM) | 5 μl |
| Reverse oligo 2121 (1 μM) | 5 μl |
| 50 mM Tris-HCl, 10 mM MgCl₂, pH 8.0 | 2 μl |
| H₂O | 8 μl |

Each of the annealing mixes were heated at 80° C. for 2 minutes on a pre-heated Hybaid OmniGene PCR machine which was subsequently turned off and allowed to cool to room temperature (about 10 min). The fragments were subsequently put on ice.

Restriction Digestion of pTrcHis-A Prokaryotic Expression Vector

The pTrcHis-A prokaryotic expression vector, cut with Ncol and BamHI restriction enzymes and gel purified, was used for cloning the prepared NZ and CZ leucine zipper coding sequences:

Restriction Digestion of pTrcHis-A Vector

| | |
|---|---|
| pTrcHis-A (1 μg/μl) | 2 μl |
| Ncol (10 U/μl) | 1 μl |
| Nhel (5 U/μl), optional | 0.5 μl |
| BamHI (20 U/μl) | 1 μl |
| 100x BSA | 0.4 μl |
| 10x NEB (New England Biolabs, NEB) BamHI buffer | 3 μl |
| H₂O | 23 μl |
| Calf intestinal phosphatase (optional, last 20 min only) | 0.5 μl |

The vector was digested for about 1 hour at 37° C. and purified by agarose gel electrophoresis. The desired vector fragment was recovered from the gel using the QIAquick Gel Extraction kit (spin columns) from Qiagen and recovered in 50 μl of elution buffer. Nhel, which cuts between NcoI and BamHI, was included to minimise the amounts of uncut and self-ligating vector.

Ligation and Transformation of Annealed NZ Oligo Pairs

Each of the three NZ annealing mixtures 1-3 was diluted 50-fold (1 μl of mixture in 50 μl of H₂O) and mixed and ligated into the cut vector as follows (three hours at 20-24° C.):

Ligation of NZ Zipper Fragments into pTrcHis-A Vector

| | |
|---|---|
| Annealing mix 1 | 1 μl |
| Annealing mix 2 | 1 μl |
| Annealing mix 3 | 1 μl |
| 10x T4 DNA ligase buffer (New England Biolabs) | 1 μl |
| T4 DNA ligase (400 U/μl, New England Biolabs) | 0.5 μl |
| pTrcHis-A (Ncol + BamHI cut) | 0.5 μl |
| H₂O | 5 μl |

Alternatively, the fragments in NZ annealing mixes 1, 2, and 3 can be ligated in absence of vector and purified by agarose gel electrophoresis before being ligated into the Ncol-BamHI cut vector. The annealed and ligated oligo nucleotides from annealing mixes 1-3 had single stranded terminal overhangs that were compatible with the overhangs that were generated by Ncol and BamHI restriction digestion of pTrcHis-A. After ligation of the fragment into cut pTrcHis-A, the Ncol and BamHI sites were regenerated.

Following ligation into the vector, 2 μl of the ligation mixture was transformed into 50 μl of One Shot TOP10 chemically competent E. coli cells (Invitrogen) following the manufacturers protocol. The ligation can be performed using different amounts or volumes fragments and buffers. The inserted DNA sequence (SEQ ID NO: 7) and the encoded NZ zipper peptide (SEQ ID NO: 8) are as follows:

```
      M A G G T G S G A L K K E L Q A N K K E
CCATGGCCGGTGGTACCGGTTCCGGTGCCCTGAAGAAGGAGCTGCAGGCCAACAAGAAGGAG
  L A Q L K W E L Q A L K K E L A Q * D
CTGGCCCAGCTGAAGTGGGAGCTGCAGGCCCTGAAGAAGGAGCTGGCCCAGTAGGATCC
```

The Gly-Gly-Thr-Gly-Ser-Gly amino acid sequence in the terminus is part of the linker sequence that was inserted between the NZ zipper peptide and the N-terminal fragments of EGFP (NtermEGFP). The zipper sequence in the NtermEGFP-NZ fusion protein is also Gly-Gly-Thr-Gly-Ser-Gly with the Gly-Gly-Thr-Gly coding sequence being repeated in the NtermEGFP reverse amplification primers 2129, 2130, and 2131 (Table 3). Underlined are the unique Ncol (CCATGG), Agel (ACCGGT) and BamHI (GGATCC) sites used for cloning of the zipper peptide into pTrcHis-A and the NtermEGFP-NZ fragments into the NZ zipper vector PS1515 (see below). The asterisk (*) shows a stop codon.

Ligation and Transformation of Annealed CZ Oligo Pairs

Each of the three CZ annealing mixtures 4-6 was diluted 50-fold (1 μl of mixture in 50 μl of H₂O) and mixed as follows:

Ligation of CZ Zipper Fragments into pTrcHis-A Vector

| | |
|---|---|
| CZ annealing mix 1 | 1 μl |
| CZ annealing mix 2 | 1 μl |
| CZ annealing mix 3 | 1 μl |
| 10x T4 DNA ligase buffer (New England Biolabs) | 1 μl |
| T4 DNA ligase (400 U/μl, New England Biolabs) | 0.5 μl |
| pTrcHis-A (Ncol + BamHI cut) | 0.5 μl |
| H₂O | 5 μl |

Alternatively, the fragments in CZ annealing mixes 1, 2, and 3 can be ligated in absence of vector and purified by agarose gel electrophoresis before being ligated into the Ncol-BamHI cut vector. The annealed and ligated oligo nucleotides from annealing mixes 1-3 had single stranded terminal overhangs that were compatible with the overhangs that were generated by Ncol and BamHI restriction digestion of pTrcHis-A. After ligation of the fragment into cut pTrcHis-A, the Ncol and BamHI sites were regenerated.

Following ligation into the vector, 2 μl of the ligation mixture were transformation into 50 μl of One Shot TOP10 chemically competent E. coli cells (Invitrogen) following the manufacturers protocol. The ligation can be performed using different amounts or volumes fragments and buffers. The inserted DNA sequence (SEQ ID NO: 9) and the encoded CZ zipper peptide (SEQ ID NO: 10) are as follows:

```
      M A S E Q L E K K L Q A L E K K L A Q L
CCATGGCCAGCGAGCAGCTGGAGAAGAAGCTGCAGGCCCTGGAGAAGAAGCTGGCCCAGCTG
  E W K N Q A L E K K L A Q G G T G *
GAGTGGAAGAACCAGGCCCTGGAGAAGAAGCTGGCCCAGGGCGGCACCGGTTAGGATCC
```

The Gly-Gly-Thr-Gly amino acid sequence in the terminus is part of the linker sequence that was inserted between the CZ zipper peptide and the C-terminal fragments of EGFP (CtermEGFP). The zipper sequence in the CZ-CtermEGFP fusion protein is also Gly-Gly-Thr-Gly with the Thr-Gly coding sequence being repeated in the CtermEGFP forward amplification primers 2133, 2134, and 2135 (Table 3). Underlined are the unique Ncol (CCATGG), Agel (ACCGGT) and BamHI (GGATCC) sites used for cloning of the zipper peptide into pTrcHis-A and the CZ-CtermEGFP fragments into the CZ zipper vector PS1516 (see below). The asterisk (*) shows a stop codon.

### Example 3

E. coli Colony PCR Screen, Plasmid Miniprep and DNA Sequencing

The transformed bacteria were plated on Luria Broth (LB) agar plates containing 100 μg/ml of carbenicillin as selection (present in used E. coli media). To quickly identify transformants containing the desired NZ or CZ constructs, colony PCR screening was performed using oligos 2110 (5′ forward NZ oligo) and 2115 (3′ reverse NZ oligo) or using oligos 2116 (5′ forward CZ oligo) and 2121 (3′ reverse CZ oligo):

Per Sample (15 μl Reaction Volume)

| | |
|---|---|
| 10x Taq polymerase buffer (Perkin Elmer) | 1.5 μl |
| dNTP (5 mM nucleotide, each) | 0.3 μl |
| 50 mM MgCl₂ | 0.6 μl |
| Dimethyl sulphoxide (DMSO) | 0.3 μl |
| Taq polymerase (Perkin Elmer) | 0.2 μl |
| 5′ forward primer (10 μM) | 0.5 μl |
| 3′ reverse primer (10 μM) | 0.5 μl |
| H₂O | 6.1 μl |
| Transformant resuspended in H₂O | 5.0 μl |

Cycling Parameters (RoboCycler Gradient 96, Stratagene)

Initial denaturation at 94° C. for 3 min followed by 25 cycles of (all steps of 1 min):

Denaturation at 94° C., primer annealing at 53° C. and primer extension at 72° C.

Finally, an additional extension step at 72° C. was included (5 min).

16 NZ transformants and 16 CZ transformants were screened. PCR fragments having the expected product sizes of about 120 base pairs were amplified from 14 NZ clones and 15 CZ clones, as determined by agarose gel electrophoresis analysis.

Three of the positive colonies were picked from each transformation (NZ and CZ) and used to inoculate 5 ml of liquid LB medium. After culturing at 37° C. over night, plasmid DNA was purified by mini preparations using the QIAprep kit from Qiagen.

Plasmids containing correct NZ (PS1515) or CZ (PS1516) fragment inserts were identified by DNA sequencing on an ABI PRISM model 377 DNA sequencer using forward sequencing primer 1282.

### Example 4

Prokaryotic Expression Vectors Encoding Fusion Proteins of EGFP Fragment and Zipper

The DNA sequences encoding the NZ and CZ zippers in the prokaryotic expression vectors PS1515 and PS1516, respectively, can be fused to DNA sequences encoding desired EGFP fragments (N-terminal fragments of EGFP are called NtermEGFP and C-terminal fragments of EGFP are called CtermEGFP) or other fragments using the unique Agel restriction sites appropriately located in linker sequences in either the 5′ end (as in the NZ vector PS1515) or in the 3′ end (as in the CZ vector PS1516) of the leucine zipper coding sequence in combination with either of the unique Ncol or BamHI sites used for cloning the zipper coding fragments (DNA and amino acid sequences are shown above). The general structures of the fusion protein coding sequences are shown in FIG. 1.

For example, to prepare a prokaryotic expression vector encoding a fusion protein consisting of NZ zipper N-terminally fused to an NtermEGFP fragment (that is, fused to the C terminal of the NtermEGFP fragment), e.g. residues 1-172 (NtermEGFP172), this region of the EGFP coding sequence in the commercial expression vector pEGFP-C1 (Clontech) was amplified by PCR using forward oligo 2128 (containing a unique Ncol site) and reverse oligo 2131 (containing a unique Agel site) in accordance with Table 3.

Per Sample (50 μl Reaction Volume)

| | |
|---|---|
| 10x Pfu polymerase buffer (Stratagene) | 5.0 μl |
| dNTP (5 mM nucleotide, each) | 1.0 μl |
| Pfu Hot Start polymerase (Stratagene) | 1.0 μl |
| 5′ forward primer (10 μM) | 1.0 μl |
| 3′ reverse primer (10 μM) | 1.0 μl |
| pEGFP-C1 vector (10 ng/μl) | 2.0 μl |
| H₂O | 39.0 μl |

Cycling Parameters (Hybaid OmniGene PCR Machine)

Initial denaturation at 94° C. for 3 min followed by 25 cycles of (all steps of 1 min):

Denaturation at 94° C., primer annealing at 53° C. and primer extension at 72° C.

Finally, an additional extension step at 72° C. was included (5 min).

The PCR fragment encoding the desired EGFP fragment, e.g. the above mentioned fragment composed of residues 1-172, with appropriately engineered terminal restriction sites contained in the primer sequences was then gel purified as described above cut with Ncol and Agel or Agel and BamHI and ligated into the constructed NZ or CZ prokaryotic leucine zipper expression vectors PS1515 or PS1516 cut with the same enzymes and gel purified:

Restriction Digestion of NtermEGFP and CtermEGFP PCR Fragments

| EGFP fragment (gel purified) | 26 μl |
|---|---|
| Ncol (10 U/μl) or BamHI (20 U/μl) | 0.5 μl |
| Agel (10 U/μl) | 1.0 μl |
| 10x New England Biolabs buffer 2 | 3 |

Restriction Digestion of NZ (PS1515) and CZ (PS1516) Vectors

| Vector (1 μg/μl) | 1.0 μl |
|---|---|
| Ncol (10 U/μl) or BamHI (20 U/μl) | 0.33 μl |
| Agel (10 U/μl) | 0.66 μl |
| 10x New England Biolabs buffer 2 | 1 |
| H₂O | 7 |

All enzymes were from New England Biolabs. The DNA preparations were digested for 1 hour at 37° C. and gel purified.

Ligation of EGFP Fragments into Cut PS1515 or PS1516 Vector

| | |
|---|---|
| Cut and purified vector | 2 μl |
| Cut and purified NtermEGFP or CtermEGFP | 4 μl |
| fragment | |
| 10x T4 DNA ligase buffer (New England Biolabs) | 1 μl |
| T4 DNA ligase (400 U/μl, New England Biolabs) | 0.5 μl |
| H₂O | 2.5 μl |

Ligation proceeded for 30 min at 22° C. after which 2 μl of each ligation mixture were transformed into 50 μl of One Shot TOP10 chemically competent E. coli cells (Invitrogen). The transformed cells were plated on LB plates containing carbenicillin and plasmids were prepared from two colonies from each transformation as described above.

### Example 5

EGFP Based Bimolecular Fluorescence Complementation in E. coli

Plasmids that expressed functional NtermEGFP-NZ or CZ-CtermEGFP complementation constructs were identified by co-transforming 10 μl of One Shot TOP10 chemically competent E. coli cells (Invitrogen) with 1 μl of each of appropriately matched NtermEGFP-NZ or CZ-CtermEGFP plasmids (i.e., plasmids that express EGFP fragments, said fragments are truncated after (NtermEGFP fragments) or before (CtermEGFP fragments) the same splitting site and plating the co-transformed cells on LB plates containing carbenicillin and 5 mM of isopropyl-β-thiogalactoside (IPTG).

The transformed cells were grown over night at 37° C. E. coli colonies that were green fluorescent because of EGFP based bimolecular fluorescence complementation were visible on the agar plate without magnification about 10-20 hours after transfection (the fluorescence developed further during storage of the plates at 5° C. for one or more days) when illuminated with a blue light source (Fiberoptic-Heim LQ2600) and viewed through yellow filter glasses.

Functional complementation was clearly visible in cells co-transformed with complementation constructs based on splits between either residues 157 and 158 or between residues 172 and 173 and the DNA sequences of expression vectors that produced functional NtermEGFP-NZ or CZ-CtermEGFP complementation fragments (named PS1594, PS1595, PS1596, PS1597, see Table 4) were verified by DNA sequencing using primer 1282 as previously described.

Surprisingly, the E. coli colonies of cells co-transformed with the vectors expressing the EGFP complementation fragments with split in the Ile171-Ser175 loop (namely between residues 172 and 173, vectors PS1595 and PS1597) were significantly more fluorescent than the colonies of cells that were co-transformed with vectors expressing EGFP complementation fragments that were split in the Ala154-Gly160 loop (namely between residues 157 and 158, vectors PS1594 and PS1596).

Functional complementation was not clearly visible in cells co-transformed with complementation constructs based on a split between residues 144 and 145. DNA sequencing confirmed that expression vectors PS1614 and PS1615 encoded the correct NtermEGFP-NZ and CZ-CtermEGFP complementation fragments, respectively.

### Example 6

Eukaryotic Expression Vectors Encoding Fusion Proteins of EGFP Fragment and Zipper

Because of the low fluorescence signal produced by the complementation fragments based on the 144/145 split fragments, only the complementation fragments that were based on splits at residues 157/158 or 172/173 were transferred to an eukaryotic expression system to permit evaluation of fragment complementation in mammalian cells.

NtermEGFP-NZ fragments in PS1596 and PS1597, and CZ-CtermEGFP fragments in PS1594 and PS1595, are flanked by an Ncol site 5′ to the start codons and a BamHI site 3′ to the stop codons. The fragments were transferred as blunt-ended Ncol/BamHI fragments into mammalian expression vectors cut with Eco47III/BamHI. To select for stable expression of both an NtermEGFP-NZ and a CZ-CtermEGFP expressing plasmid, the expression vectors for NtermEGFP-NZ fragments and CZ-CtermEGFP fragments contain selection markers for neomycin/geneticin/G418 and zeocin, respectively.

Plasmids PS1594, PS1595, PS1596, and PS1597 were cut with Ncol restriction enzyme, blunt-ended with Klenow fragment, gel purified, cut with BamHI and gel purified as described below.

Restriction digestion of NtermEGFP-NZ and
CZ-CtermEGFP prokaryotic expression vectors

| | |
|---|---|
| PS1594, PS1595, PS1596, or PS1597 (1 μg/μl) | 1 μl |
| Ncol (10 U/μl, from New England Biolabs) | 1 μl |
| 10x buffer 4 (NEB) | 3 μl |
| H₂O | 25 μl |

The plasmids were digested for about 1 hour at 37° C. 1 μl of 1 mM dNTP mix and 1 unit of Klenow fragment (New England Biolabs) were added and the reactions were incubated 30 minutes at room temperature. The linear plasmid fragments were purified by agarose gel electrophoresis and recovered from the gel using the QIAquick Gel Extraction kit (spin columns) from Qiagen and recovered in 50 μl of elution buffer. 5 μl BamHI buffer (New England Biolabs) and 10 units BamHI enzyme were added. The plasmids were digested for about 1 hour at 37° C. The desired plasmid fragments were purified by agarose gel electrophoresis and recovered from the gel using the QIAquick Gel Extraction kit (spin columns) from Qiagen and recovered in 50 μl of elution buffer.

To stably co-express NtermEGFP-NZ and CZ-CtermEGFP fragments in the same mammalian cell, mammalian expression vectors carrying different selection markers were required. To obtain this, the kanamycin/neomycin selection marker on the expression vector pEGFP-C1 was replaced with a zeocin resistance marker resulting in the plasmid referred to as PS0609.

Replacement of Kanamycin/Neomycin Marker on pEGFP-C1 with Zeocin Marker.

pEGFP-C1 was digested with AvrII, which excises the kanamycin/neomycin selection marker, and following gel purification, the vector fragment was ligated with an approximately 0.5 kbp AvrII fragment encoding zeocin resistance. This fragment was isolated by PCR amplification of the zeocin selection marker on plasmid pZeoSV (Invitrogen) using primers 9655 and 9658 (see Table 2). Both primers contain AvrII cloning sites and flank the zeocin resistance gene on plasmid pZeoSV including its E. coli promoter. The top primer 9658 spans the AseI site at the beginning of zeocin, which can be used to determine the orientation of the AvrII insert relative to the SV40 promoter which drives resistance in mammalian cells. The resulting plasmid is referred to as PS0609.

Plasmids pEGFP-C1 (Clontech) and its zeocin-resistant derivative PS0609 were cut with Eco47III restriction enzyme, gel purified, cut with BamHI and gel purified as described below. These steps excise EGFP and leave the rest of the vectors intact.

Restriction digestion of eukaryotic expression vectors

| | |
|---|---|
| pEGFP-C1 or PS0609 DNA (1 μg/μl) | 0.5 μl |
| Eco47III (10 U/μl, from Promega) | 1 μl |
| 10x buffer D (Promega) | 3 μl |
| H₂O | 25.5 μl |

The plasmids were digested for about 1 hour at 37° C. The linear plasmid fragments were purified by agarose gel electrophoresis and recovered from the gel using the QIAquick Gel Extraction kit (spin columns) from Qiagen and recovered in 50 μl of elution buffer. 5 μl BamHI buffer (New England Biolabs) and 10 units BamHI enzyme were added. The plasmids were digested for about 1 hour at 37° C. The desired vector fragments were purified by agarose gel electrophoresis and recovered from the gel using the QIAquick Gel Extraction kit (spin columns) from Qiagen and recovered in 50 μl of elution buffer.

Ligation of NtermEGFP-NZ fragments into pEGFP-C1
and CZ-CtermEGFP fragments into PS0609

| | |
|---|---|
| Cut and purified vector fragment | 1 μl |
| Cut and purified NtermEGFP-NZ or CZ-CtermEGFP | 3 μl |
| fragment | |
| 10x T4 DNA ligase buffer (New England Biolabs) | 1 μl |
| T4 DNA ligase (400 U/μl, New England Biolabs) | 0.5 μl |
| H2O | 5 μl |

Ligation reactions were incubated at 16° C. overnight. 3 μl were transformed into One Shot TOP10 chemically competent E. coli cells (Invitrogen) and transformants were selected on imMedia with kanamycin or imMedia with zeocin (both from Invitrogen) for pEGFP-C1 and PS0609 derivatives, respectively.

4 transformants from each transformation plate were picked in imMedia medium with appropriate selection (kanamycin or zeocin) and grown at 37 degrees C. for 6 hours.

Plasmid DNA was isolated by the QIAprep spin column method (Qiagen) and analysed by restriction digests with AseI and MluI. The DNA sequences of the inserts were finally verified by sequencing as described above. The resulting plasmids were named PS1557, PS1558, PS1559, and PS1560 (Table 4).

### Example 7

EGFP Based Bimolecular Fluorescence Complementation in Mammalian Cells

To establish cells lines that express EGFP fragment/zipper fusion proteins, CHO-hIR cells were transfected with plasmid pairs resulting in two cell lines 1) CHO-hIR PS1559+PS1557, and 2) CHO-hIR PS1560+PS1558. The CHO-hIR cell line consists of CHO-K1 (ATCC CCL-61) cells that have been stably transfected with the human insulin receptor ((hIR, GenBank Acc# M10051) as described in: Hansen, B. F., Danielsen, G. M., Drejer, K., Sørensen, A. R., Wiberg, F. C., Klein, H. H., Lundemose, A. G. (1996) Sustained signalling from the insulin receptor after stimulation with insulin analogues exhibiting increased mitogenic potency. Biochem. J. April 1; 315 (Pt 1):271-279)<. The selection marker for the vector is methotrexate (MTX). The hIR expression is very stable in the CHO-hIR cells, without selection pressure, because of the insulin-sensitivity of the cell line and a very stable phenotype can be maintained without selection pressure.

Stable cells were obtained by cell growth in selection medium containing Geneticin and Zeocin.

CHO-hIR cells were transfected using Fugene (Roche) according to the manufacturer's instructions. The day after transfection, cells were examined for transient expression, split 1:10 and exposed to selection medium (growth medium supplemented with 500 μg/ml geneticin (invitrogen) and 1 mg/ml zeocin (Cayla). The cells lines were stable after 2-3 weeks of culture in selection medium.

The growth medium used was NUT.MIX F-12 (Ham's) with GLUTAMAX-1 (Gibco/Invitrogen) supplemented with 10% fetal bovine serum (JRH Biosciences) and 1% Penicillin-Streptomycin (10,000 IU/ml, Gibco/Invitrogen). The CHO-hIR cells were cultured in growth medium, and split 1:4 to 1:16 twice a week according to standard cell culture protocols. The CHO-hIR PS1559+PS1557 and CHO-hIR PS1560+PS1558 were treated likewise, except that the growth medium was supplemented with 500 μg/ml geneticin (Invitrogen) and 1 mg/ml zeocin (Cayla) at all times.

Images of three CHO-hIR cell lines separately transfected with pEGFP-C1 (expressing GFP with a short C-terminal extension), PS1559+PS1557 (expressing EGFP complementation fragments split at 157-158, NtermEGFP157-NZ+CZ-CtermEGFP158) and with PS1560+PS1558 (expressing EGFP complementation fragments split at 172-173, NtermEGFP172-NZ+CZ-CtermEGFP173) were collected 1 day, 2 days and 10 days after transfection to assess the relative brightness of cells expressing the different complementation constructs. Images were collected on a Nikon Diaphot 300 equipped for epifluorescence work. Light source for epifluorescence was a Nikon 100W Hg arc lamp, coupled to the microscope through a custom quartz fibre illuminator (TILL Photonics GmbH, Planegg, Germany). Excitation light passed through a 450-490 nm bandpass filter (Delta Light and Optics, Lyngby, Denmark) and was directed to the specimen via a Chroma 72100 505 nm cut-on dichroic mirror (Chroma Technology, Brattleboro, Vt., USA). A ×40 NA1.3 oil immersion lens was used for all images. Emitted light passed through a 540-550 bandpass filter (Chroma) to a Hammamatsu Orca ER camera. All images were collected with 50 millisecond exposure time, chosen to ensure non-saturation of images for even the brightest (EGFP-expressing) cells in each optical field (maximum pixel count<4095). Imaging software used to acquire images on this system was IPLab for Windows (Scanalytics, USA).

Presentation and Analysis of Images

The microscope images were analysed using the ImageJ software package, the public domain image analysis software written by Wayne Rasband of the US National Institute of Health (http://rsb.info.nih.gov/ij/) and the data analysis was performed in Microsoft Excel. The images shown in FIG. 2 are of fluorescent CHO-hIR cells co-transfected with different NtermEGFP-NZ and CZ-CtermEGFP expression vectors or transfected with pEGFP-C1. The images are scaled individually to visualise the cells and the fluorescence distribution within them. Because of this scaling, the relative fluorescence levels cannot be compared between the images. When the same images are scaled identically they appear as in FIG. 3 and it is apparent that the cells that are transfected with complementation constructs that are based on a split between residues 172 and 173 are significantly more fluorescent than the cells that are transfected with complementation constructs that are based on a split between residues 157 and 158. However, the cells transfected with the pEGFP-C1 construct show significantly stronger fluorescence on day 2.

The same images were analysed for background and maximum fluorescence intensities using the ImageJ software package (FIG. 4). From the figure, it is clear that a split between residues 172 and 173, and probably anywhere else in this loop, is greatly superior to a split between residues 157 and 158 and probably also to splits anywhere else in this loop.

### Example 8

Eukaryotic Expression Vectors Encoding EYFP and EYFP Variant F64L Fragment/Zipper Fusion Proteins

Mutagenesis of the eukaryotic NtermEGFP-NZ expression vectors PS1559 (NtermEGFP157-NZ) and PS1560 (NtermEGFP172-NZ) into the corresponding N-terminal EYFP (SEQ ID NO: 5) fragment (NtermEYFP-NZ) variants and mutagenesis of the eukaryotic CtermEGFP expression vectors PS1557 (CZ-CtermEGFP158) and PS1558 (CZ-CtermEGFP173) into the corresponding C-terminal EYFP fragment (CZ-CtermEYFP) variants was accomplished by site directed mutagenesis using the QuickChange kit and by following the manufacturers instructions (Stratagene). Primers 2333 and 2334 were used to convert expression vectors PS1559 (NtermEGFP157-NZ) and PS1560 (NtermEGFP172-NZ) into N-terminal EYFP fragment expression vectors PS1639 (NtermEYFP157-NZ) and PS1642 (NtermEYFP172-NZ). The introduced mutations were: L64F:T65G:V68L:S72A. Furthermore, primers 2335 and 2336 were used to convert expression vectors PS1559 (NtermEGFP157-NZ) and PS1560 (NtermEGFP172-NZ) into F64L mutated N-terminal EYFP fragment expression vectors PS1640 (NtermE[F64L]YFP157-NZ) and PS1641 (NtermE[F64L]YFP172-NZ). The introduced mutations were: T65G:V68L:S72A. Accordingly, the expressed NtermEYFP fragments have the following amino acid sequences (only residues 64-72 are shown):

| | 64 | 65 | 66 | 67 | 68 | 69 | 70 | 71 | 72 |
|---|---|---|---|---|---|---|---|---|---|
| NtermEGFP | L | T | Y | G | V | Q | C | F | S |
| (template) | | | | | | | | | |
| NtermEYFP | F | G | Y | G | L | Q | C | F | A |
| (L64F:T65G:V68L:S72A) | | | | | | | | | |
| NtermE[F64L]YFP | L | G | Y | G | L | Q | C | F | A |
| (T65G:V68L:S72A) | | | | | | | | | |

Finally, primers 2337 and 2338 were used to convert expression vectors PS1557 (CZ-CtermEGFP158) and PS1558 (CZ-CtermEGFP173) into C-terminal EYFP fragment expression vectors PS1637 (CZ-CtermEYFP158) and PS1638 (CZ-CtermEYFP173) by introducing a T203Y mutation. All sequences were verified by DNA sequencing of the vectors and all primer sequences are shown in Table 2.

### Example 9

EGFP Based Bimolecular Fluorescence Complementation in Mammalian Cells

The constructed EYFP based split fluorescent protein expression vectors PS1637 to PS1642 described above were investigated in mammalian cells in parallel with the EGFP based split fluorescent protein expression vectors PS1557 to PS1560 described in Example 7 and using the same experimental set-up (including the same filter set) and procedures (including the image analysis procedure) except that all images were produced using 10 ms exposure times instead of 50 ms exposure times, because of the increased brightness of the probes, and a 20×objective was used instead of a 40×objective to image more cells. Other appropriate filter sets could have been used. The images are taken the day after transfection (day 1).

It is apparent from the identically scaled fluorescence images of the transfected cells (FIG. 6) that the split site between residues 172 and 173 is again shown to be superior to the split site between residues 157 and 158. Furthermore, it is apparent that complementation based on EYFP fragments is superior to complementation based on EGFP fragments. Surprisingly, introduction of the F64L mutation from EGFP into the N-terminal EYFP fragments further greatly enhanced the fluorescence of the complementing fragments. As can be seen from the images, the positive effects of using the optimal splitting site (between residues 172 and 173) using the optimal fluorescent protein colour variant (EYFP) and introducing the F64L folding mutation into the NtermEYFP fragment, are additive. Quantification of these observation was done by analysing the images shown in FIG. 6 and the numeric out-put is presented in FIG. 7.

| | |
|---|---|
| Good | Better |

Effects of colour (yellow better):

| | | |
|---|---|---|
| EGFP | vs | EYFP |
| NtermEGFP157-NZ + | vs | NtermEYFP157-NZ + |
| CZ-CtermEGFP158 | | CZ-CtermEYFP158 |
| NtermEGFP172-NZ + | vs | NtermEYFP172-NZ + |
| CZ-CtermEGFP173 | | CZ-CtermEYFP173 |

Effects of split site (172/173 better):

| | | |
|---|---|---|
| NtermEGFP157-NZ + | vs | NtermEGFP172-NZ + |
| CZ-CtermEGFP158 | | CZ-CtermEGFP173 |
| NtermEYFP157-NZ + | vs | NtermEYFP172-NZ + |
| CZ-CtermEYFP158 | | CZ-CtermEYFP173 |
| NtermE[F64L]YFP157-NZ + | vs | NtermE[F64L]YFP172-NZ + |
| CZ-CtermEYFP158 | | CZ-CtermEYFP172 |

Effects of F64L (+F64L better):

| | | |
|---|---|---|
| NtermEYFP157-NZ + | vs | NtermE[F64L]YFP157-NZ + |
| CZ-CtermEYFP158 | | CZ-CtermEYFP158 |
| NtermEYFP172-NZ + | vs | NtermE[F64L]YFP172-NZ + |
| CZ-CtermEYFP173 | | CZ-CtermEYFP173 |

It is interesting to note, that the optimal constructs (NtermE[F64L]YFP172-NZ and CZ-CtermE[F64L]YFP173) when re-assembled is nearly as intense as EYFP itself. The great increase in fluorescence intensity is important in many types of quantitative cell analyses (e.g. high through-put screening and microscopy) to increase the signal to noise rations, to facilitate detection of low amounts of probes in vivo or in vitro, etc.

Mixing NtermEYFP with CtermEGFP or NtermEGFP with CtermEYFP fragments can also produce functional fluorescent complexes, potentially of different colours (FIGS. 8 and 9). Fragments having overlapping sequences are also functional and may be very attractive in e.g. functional cloning systems where highly flexible linkers sequences are required due to the very diverse nature of the fusion partners. The overlapping fragments permit either of the fusion partners to have a long linker sequence (FIG. 8, quantified in FIG. 9).

### Example 10

Construction of PS1769, 1767, 1771, 1768

Plasmid PS1769 encodes a fusion of NtermE[F64L]YFP172 and FKBP, connected by a linker sequence GSGSGSGDITSLYKKAGST (1 letter amino acid code, SEQ ID NO: 11) derived in part from the Gateway recombination sequence.

Plasmid PS1767 encodes a fusion of NtermE[F64L]YFP172 and the FKBP binding part of FRAP, FRB (amino acids 2025-2114 of FRAP), connected by a linker sequence GSGSGSGDITSLYKKAGST (1 letter amino acid code, SEQ ID NO: 12) derived in part from the Gateway recombination sequence.

Plasmid PS1771 encodes a fusion FRB and CtermEYFP173, connected by a linker sequence DPAFLYKVVISGSGSGSG (1 letter amino acid code, SEQ ID NO: 13) derived in part from the Gateway recombination sequence.

Plasmid PS1768 encodes a fusion of FKBP and CtermEYFP173, connected by a linker sequence DPAFLYKVVISGSGSGSG (1 letter amino acid code, SEQ ID NO: 14) derived in part from the Gateway recombination sequence.

Construction of Plasmid PS1769.

Plasmid PS1769 encodes a fusion of NtermE[F64L]YFP172 and FKBP, connected by a linker sequence, under the control of a CMV promoter and with kanamycin and neomycin resistance as selectable marker in E.coli and mammalian cells, respectively.

Plasmid PS1769 was derived from plasmids PS1779 (entry clone) and PS1679 (destination vector). Plasmid PS1679 was derived from plasmids PS1672 and pEGFP-C1(Clontech). Plasmid PS1672 was derived from plasmid PS1641 described above.

Construction of Intermediate PS1672.

PS1641 was subjected to PCR with primers 2219 and 2222 (Table 2), and the ca 0.5 kb Nhe1-BamH1 fragment was ligated into pEGFP-C1 (Clontech) digested with Nhe1 and BamH1. This replaces NtermEGFP with NtermE[F64L]YFP172 followed by a linker sequence, which encodes in frame linker sequence Gly-Ser-Gly-Ser-Gly-Ser-Gly, and a unique EcoRV site just upstream of BamH1. This plasmid is called PS1672.

Construction of Destination Vector PS1679.

Plasmid PS1672 was converted into a Gateway compatible destination vector by cutting the DNA with EcoRV and ligating it with Gateway Cassette reading frame A, following the recommendations of the Gateway manufacturer (Invitrogen). This destination vector is called PS1679.

Construction of Gateway Entry Clone PS1779.

The coding sequence of FKBP (GenBank Acc no XM_{—}016660) was isolated from human cDNA using PCR and primers 2442 and 1272 (Table 2). The ca 0.4 kb product was transferred by a BP reaction into donor vector pDONR207, following the manufacturers recommendations (Invitrogen), to produce entry clone PS1779.

Finally, the expression vector PS1769 was produced by transferring FKBP from entry clone PS1779 with an LR reaction into destination vector PS1679 following the manufacturers recommendations (Invitrogen).

Construction of Plasmid PS1767.

Plasmid PS1767 encodes a fusion of NtermE[F64L]YFP172 and the FKBP binding part of FRAP, FRB (amino acids 2025-2114 of FRAP), connected by a linker sequence, under the control of a CMV promoter and with kanamycin and neomycin resistance as selectable marker in E.coli and mammalian cells, respectively.

Plasmid PS1767 was derived from plasmids PS1781 (entry clone) and PS1679 (destination vector). Plasmid PS1679 was constructed as described above.

Construction of Gateway Entry Clone PS1781.

The FKBP binding part of FRAP (amino acids 2025-2114, Gen Bank Acc no XM_{—}001528) was isolated from human cDNA using PCR and primers 2444 and 1268 (Table 2). The ca 0.3 kb product was transferred by a BP reaction into donor vector pDONR207, following the manufacturers recommendations (Invitrogen), to produce entry clone PS1781.

Finally, the expression vector PS1767 was produced by transferring FRB from entry clone PS1781 with an LR reaction into destination vector PS1679 following the manufacturers recommendations (Invitrogen).

Construction of Plasmid PS1771.

Plasmid PS1771 encodes a fusion of the FKBP binding part of FRAP called FRB (amino acids 2025-2114 of FRAP) and the C-terminal of EYFP (FRB-CtermEYFP173), connected by a linker sequence, under the control of a CMV promoter and with zeocin resistance as selectable marker in E.coli and mammalian cells.

Plasmid PS1771 was derived from plasmids PS1782 (entry clone) and PS1688 (destination vector). Plasmid PS1688 was derived from plasmids PS1674 and PS609 described above. Plasmid PS1674 was derived from plasmid PS1638 described above.

Construction of Intermediate PS1674.

PS1638 was subjected to PCR with primers 2225 and 2132 (Table 2), and the ca 0.25 kb Nhe1-BamH1 fragment was ligated into PS609 digested with Nhe1 and BamH1. This replaces EGFP with EYFP(173-238) preceded by a linker sequence, which encodes in frame linker sequence Gly-Ser-Gly-Ser-Gly-Ser-Gly, and a unique EcoRV site just downstream of Nhe1. This plasmid is called PS1674.

Construction of Destination Vector PS1688.

Plasmid PS1674 was converted into a Gateway compatible destination vector by cutting the DNA with EcoRV and ligating it with Gateway Cassette reading frame A, following the recommendations of the Gateway manufacturer (Invitrogen). This destination vector is called PS1688.

Construction of Gateway Entry Clone PS1782.

The FKBP binding part of FRAP (GenBank Acc no XM_{—}001528, amino acids 2025-2114) was isolated from human cDNA using PCR and primers 2444 and 2445 (Table 2). The ca 0.3 kb product was transferred by a BP reaction into donor vector pDONR207, following the manufacturers recommendations (Invitrogen), to produce entry clone PS1782.

Finally, the expression vector PS1768 was produced by transferring FRB from entry clone PS1782 with an LR reaction into destination vector PS1688 following the manufacturers recommendations (Invitrogen).

Construction of Plasmid PS1768.

Plasmid PS1768 encodes a fusion of FKBP and EYFP(173-238) (FKBP-CtermEYFP173), under the control of a CMV promoter and with zeocin resistance as selectable marker in E.coli and mammalian cells.

Plasmid PS1768 was derived from plasmids PS1780 (entry clone) and PS1688 (destination vector). Plasmid PS1688 was constructed as described above.

Construction of Gateway Entry Clone PS1780.

The coding sequence of FKBP (GenBank Acc no XM_{—}016660) was isolated from human cDNA using PCR and primers 2442 and 2443 (Table 2). The ca 0.4 kb product was transferred by a BP reaction into donor vector pDONR207, following the manufacturers recommendations (Invitrogen), to produce entry clone PS1780.

Finally, the expression vector PS1768 was produced by transferring FKBP from entry clone PS1780 with an LR reaction into destination vector PS1688 following the manufacturers recommendations (Invitrogen).

### Example 11

Construction of an Inducible Interaction System Using the GFP Complementation Method that Demonstrates Utility of the Method in Screening for Compounds that Inhibit Protein-Protein Interactions.

The immunosuppressive compound rapamycin binds to FK506 binding protein (FKBP) and simultaneously to the large PI3Kinase homolog FRAP (also known as mTOR or RAFT), and thus serves as an heterodimeriser compound for these two proteins. To use rapamycin to induce heterodimers between proteins of interest, one of the proteins is fused to FKBP domains, and the other to a 90 amino acid portion of FRAP, termed FRB, that is sufficient for the binding the FKBP-rapamycin complex (Chen et al, PNAS 92, 4947 (1995)). In this example fusions of FRB and FKBP were made to complementary halves of split-EYFP (which included the F64L mutation in the EYFP(1-172) sequence (NtermE[F64L]YFP172)), so that the complementation reaction could be controlled by addition of rapamycin.

This example demonstrates that a model GFP complementation system using components which can be made to interact conditionally does respond as expected in a dose-dependent manner to the interaction stimulus. The example also provides information about the rate of fluorescence development for the E[F64L]YFP complementation system. Further it demonstrates that the system can be used to detect compounds that will block the interaction of proteins fused to the complementary halves of the E[F64L]YFP complementation system.

The following fusion constructs were made as described in Example 10:

NtermE[F64L]YFP172-FKBP=plasmid code PS1769

FRB-CtermEYFP173=PS1771

NtermE[F64L]YFP172-FRB=PS1767

FKBP-CtermEYFP173=PS1768

Probes were co-transfected in pairs into CHO-hIR cells (supra), PS1769 with PS1771 and PS1767 with PS1768, using the transfection agent FuGENE™ 6 (Boehringer Mannheim Corp, USA) according to the method recommended by the suppliers. Cells were cultured in growth medium (HAM's F12 nutrient mix with Glutamax-1, 10% foetal bovine serum (FBS), 100 μg penicillin-streptomycin mixture ml⁻¹ (GibcoBRL, supplied by Life Technologies, Denmark)). Transfected cells were cultured in this medium, with the addition of two selection agents appropriate to the plasmids being used, being 1 mg/ml zeocin plus 0.5 mg/mlG418 sulphate. Cells were cultured at 37° C. in 100% humidity and conditions of normal atmospheric gases supplemented with 5% CO₂.

After 10 to 12 days culture in the continuous presence of the selection agents, the resulting cell lines were judged to be stably transfected. For fluorescence microscopy, aliquots of cells were transferred to Lab-Tek chambered cover glasses (Nalge Nunc International, Naperville USA) and allowed to adhere for at least 24 hours to reach about 80% confluence. Images were routinely collected using a Nikon Diaphot 300 inverted fluorescence microscope (Nikon Corp., Tokyo, Japan) using ×20 (dry) and/or ×40 (oil immersion) objectives and coupled to a Orca ER charged coupled device (CCD) camera (Hammamatsu Photonics K. K., Hammamatsu City, Japan). The cells are illuminated with a 100 W HBO arc lamp via a 470±20 nm excitation filter, a 510 nm dichroic mirror and a 515±15 nm emission filter for minimal image background. Image collection, subsequent measurement and analysis of fluorescence intensity were all controlled by IPLab Spectrum for Windows software (Scanalytics, Fairfax, Va. USA).

Cells were also grown for 16 hours from a seeding density of approximately 1.0×10⁵ cells per 400 μL in plastic 96-well plates (Polyfiltronics Packard 96-View Plate or Costar Black Plate, clear bottom; both types tissue culture treated) both for imaging purposes and for measurements of fluorescence intensity in fluorescence plate readers. Prior to experiments, the cells are cultured over night without selection agent(s) in HAM F-12 medium with glutamax, 100 μg penicillin-streptomycin mixture ml⁻¹ and 10% FBS. This medium has low auto fluorescence enabling fluorescence measurements on cells straight from the incubator. For endpoint measurements, cells in plates were routinely fixed with 4% formaldehyde in phosphate buffered saline (PBS)+10 μM Hoechst 22538 for 10 minutes, followed by 3 wash steps using PBS. The use of the nuclear dye Hoechst 22538 enables correction of the EYFP fluorescence signal from each well for cell density. Plates prepared in this way were measured on a Fluoroskan Ascent CF plate reader (Labsystems, Finland) equipped with appropriate filter sets (EYFP: excitation 485 nm, emission 527 nm; Hoechst 22538: excitation 355 nM, emission 460 nm).

Both cell lines CHO-hIR [PS1769+PS1771] and CHO-hIR [PS1767+PS1768] responded to rapamycin with a substantial increase in EYFP fluorescence after several hours incubation, as expected (FIG. 10). At the starting condition for these cells (t=0), fluorescence is barely visible in most cells, although it was noted that some cells (<5%) in the population had low, but appreciable, fluorescence before treatment (FIG. 10a). After 4 hours (FIG. 10b) many cells (approximately 40%) had developed significantly greater EYFP fluorescence throughout the cytoplasmic and nuclear compartments. After 16 hours (FIG. 10c) the response per cell had increased further and encompassed a larger proportion of the cell population (approximately 70%). Results were essentially identical for the second cell line CHO-hIR [PS1769+PS1771].

The graph in FIG. 11 shows the rate of development of cellular EYFP fluorescence following rapamycin treatment of the CHO-hIR [PS1767+PS1768] line. Cells were treated in 96-well plates with 3 μM rapamycin and the fluorescence measured at various times. Treatment and measurements were made with the cells growing in HAM's medium+10% FBS, and fluorescence measurements were corrected for the background fluorescence from this medium. The graph demonstrates that the half-time for development of fluorescence is approximately 5 hours. The rate of development of fluorescence includes time for interaction between FKBP and FRB mediated by the dimeriser rapamycin, plus the time for annealing of the EYFP moieties, and the (presumably much longer) time needed for maturation of the fluorophore within the successfully annealed EYFP protein.

FIG. 12 is a response curve to different rapamycin doses for the CHO-hIR [PS1769+PS1771] cell line. Cells were cultured in 96-well plates, treated with various rapamycin doses for 16 hours, then fixed and stained with Hoechst prior to determination of EYFP fluorescence/cell (arbitrary units) on the Ascent plate reader. Values are corrected for PBS background as well as cell number. The cell line shows approximately a 3-fold increase in the EYFP intensity/cell over the dose range of rapamycin used in this experiment.

One way to increase the dynamic range of the response, and to decrease the inherent EYFP background signal from these cell lines, is to remove the fraction of cells that are EYFP bright prior to rapamycin stimulation. This is easily accomplished through fluorescence activated cell sorting (FACS) methods. Each of the cell lines were sorted by this method into 3 groups: (i) most green group (ii) medium to low-green group and (iii) black group. The ‘most green’ was discarded in each case, while the other 2 groups were cultured for further use. FIG. 13(a) and FIG. 13(b) show the improved response to 100 nM rapamycin of cell line CHO-hIR [PS1767+PS1768] after the sorting procedure.

FIGS. 14(a) and (b) show the response of the ‘medium to low-green’ and ‘black’ FACS groups (respectively) derived from the CHO-hIR [PS1767+PS1768] parent line. Dose response to rapamycin was measured after 7 hours (a) and 30 hours (b) for each cell line. Values for fluorescence have been corrected for plate & medium background. Increase in EYFP fluorescence is better than 20-fold the unstimulated value in each case. Unexpectedly, the absolute fluorescence signal does not appear to change significantly between 7 and 30 hours, although the cells are still alive during this period. Furthermore, the dose-response curves at 7 and 30 hours for each cell line are very closely similar, with an EC₅₀ of approximately 0.25 μM in the ‘medium to low-green’ group, and 0.1 μM in the ‘black’ group. This data suggest that once the dimerisation has occurred, the EYFP complements are stable within the cells for longer than 30 hours. The ‘medium to low-green’ group has a greater overall response range, reaching intensities of greater than 3-fold that of the black group at the highest rapamycin concentration. Both FACS groups have significantly lower pre-stimulation fluorescence intensities compared to the parent (non-FACS'd) lines.

FIGS. 15(a) and (b) show dose-response competition curves for FK506 versus 100 nM rapamycin in two of the FACS'd lines, CHO-hIR [PS1768+PS1767] ‘mid to low-green’ group (FIG. 15(a)) and CHO-hIR [PS1769+PS1771] ‘black’ group (FIG. 15(b)). EC₅₀ values in both cases are approximately 1.2 μM FK506. The cells were incubated overnight (16 hours) with mixtures of the two compounds, then fixed and stained with Hoechst prior to detemination of EYFP fluorescence/cell on an Ascent plate reader. Plate and solution backgrounds have been subtracted; the dashed lines on each graph indicate the prestimulated fluorescence levels for each cell line in these experiments. These results indicate that the GFP complementation method employing fusions to NtermE[F64L]YFP172 and CtermEYFP173 may be used successfully to screen for compounds that interfere with a conditional interaction between two protein components.

### FIGURE LEGENDS

FIG. 1

General structures of the fusion protein coding sequences.

FIG. 2

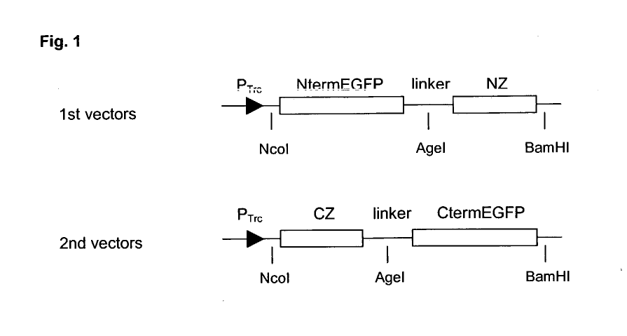

16 bit images of fluorescent CHO-hIR cells co-transfected with NtermEGFP-NZ and CZ-CtermEGFP expression vectors or transfected with pEGFP-C1 were taken and scaled individually to visualise the cells and the fluorescence distribution within them. Because of the pixel intensity scaling, the relative fluorescence levels cannot be compared among the images. The splitting sites are either between residues 157/158 (top row, plasmids PS1557 and PS1559) or between residues 172/173 (middle row, plasmids PS1558 and PS1560). The EGFP expression vector pEGFP-C1 was transfected into the cells in the bottom row. The images were taken 1 day (left column), 2 days (middle column), or 10 days (right column) after transfection. The images of the cells are representative of the cells that expressed functionally complementing fragments.

FIG. 3

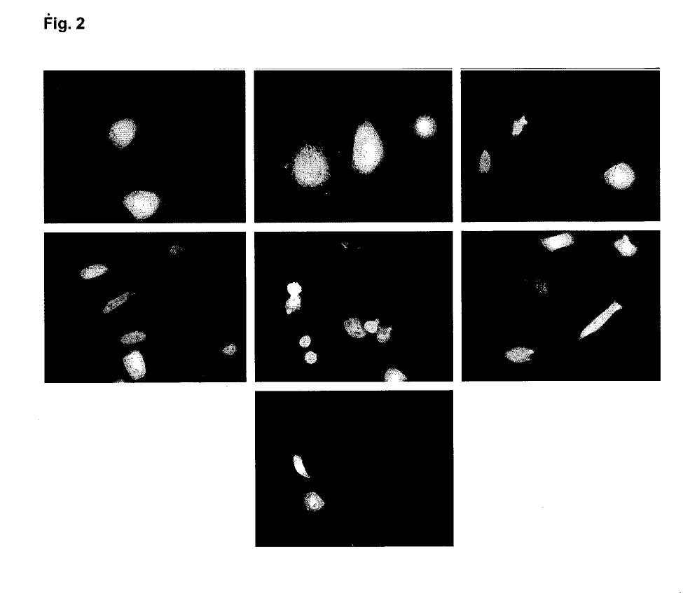

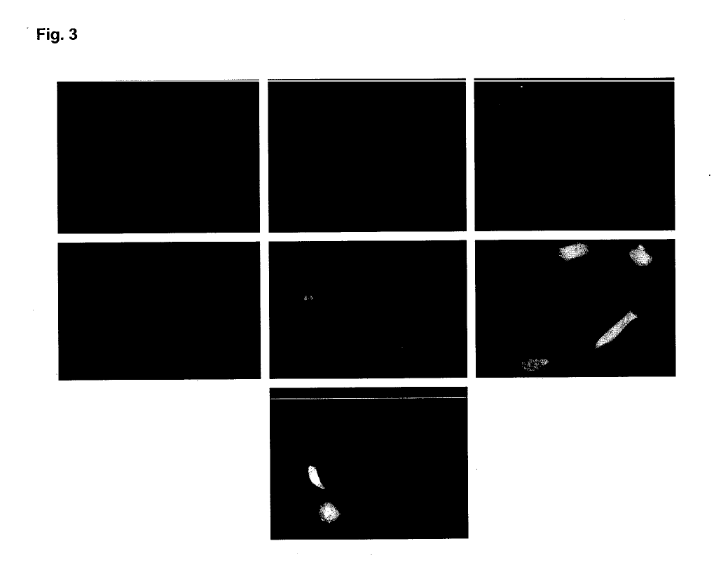

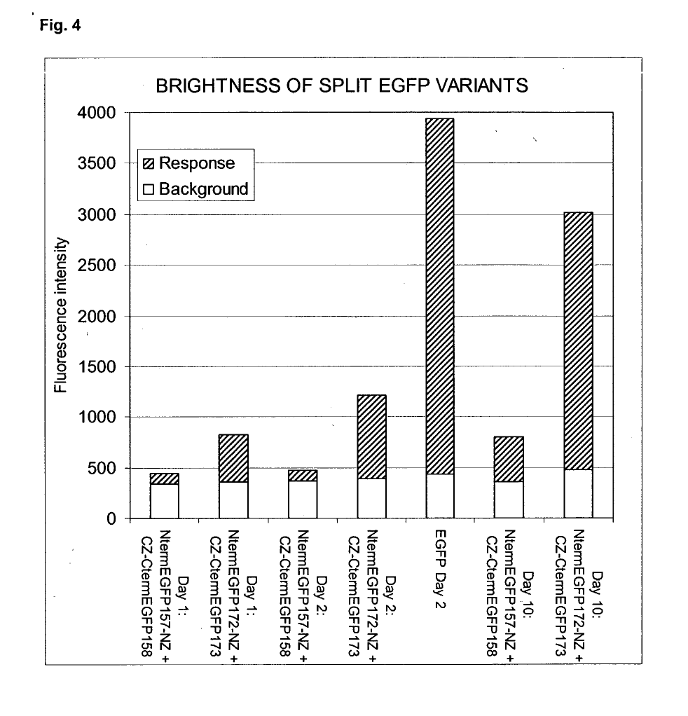

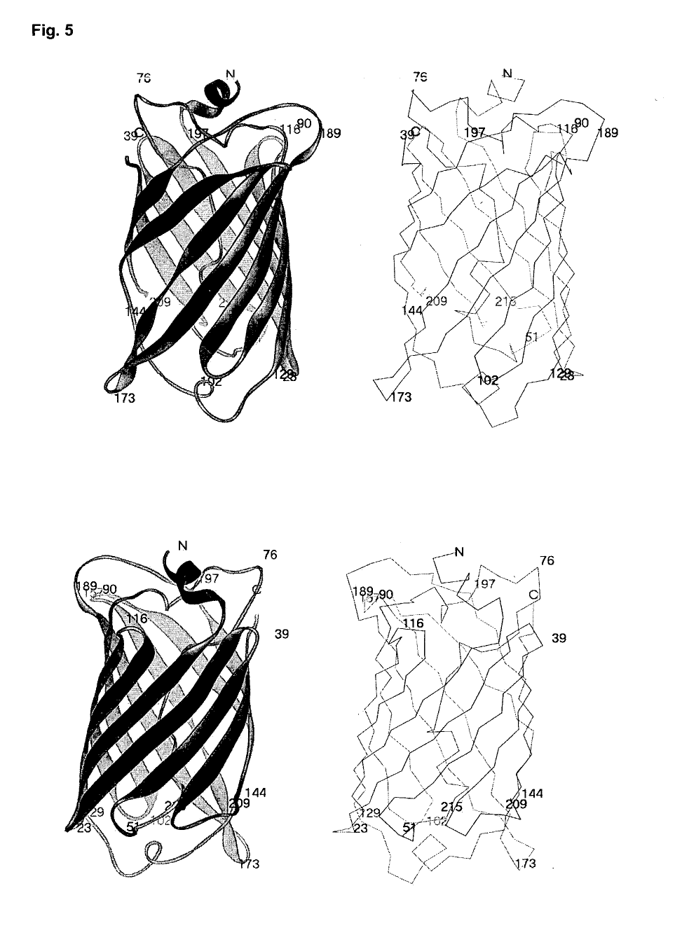

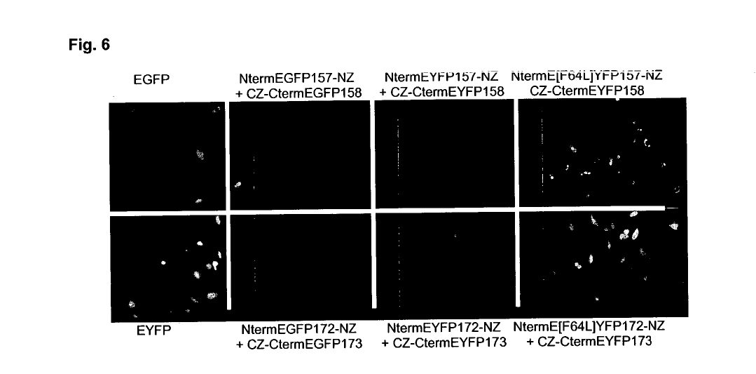

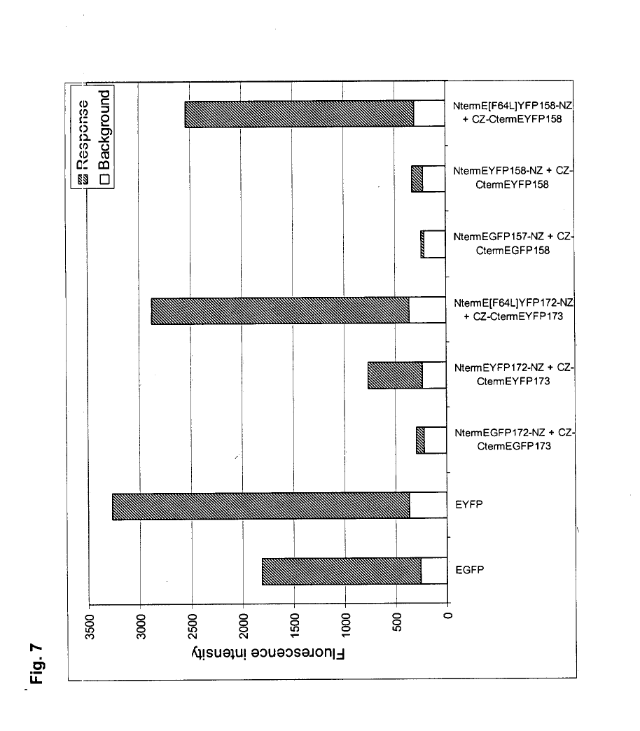

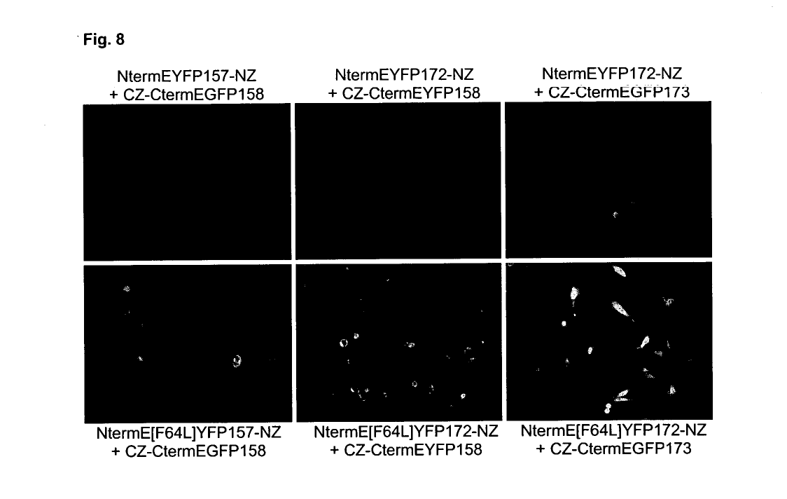

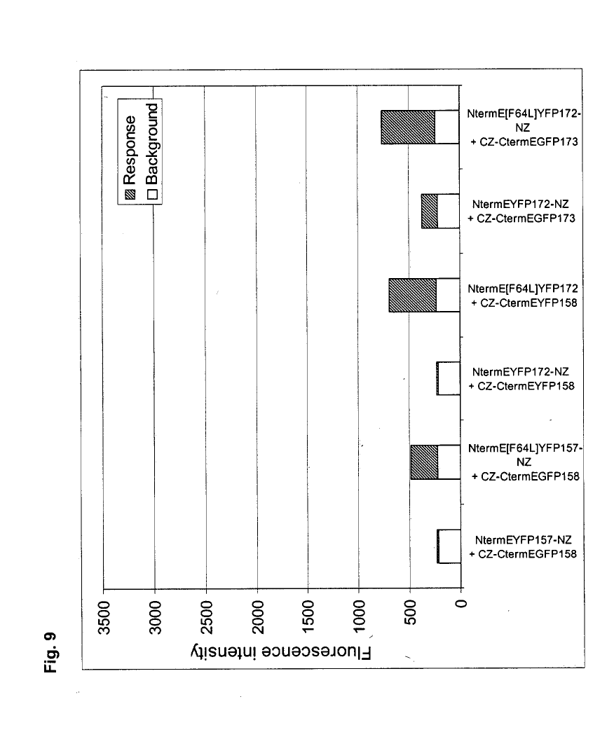

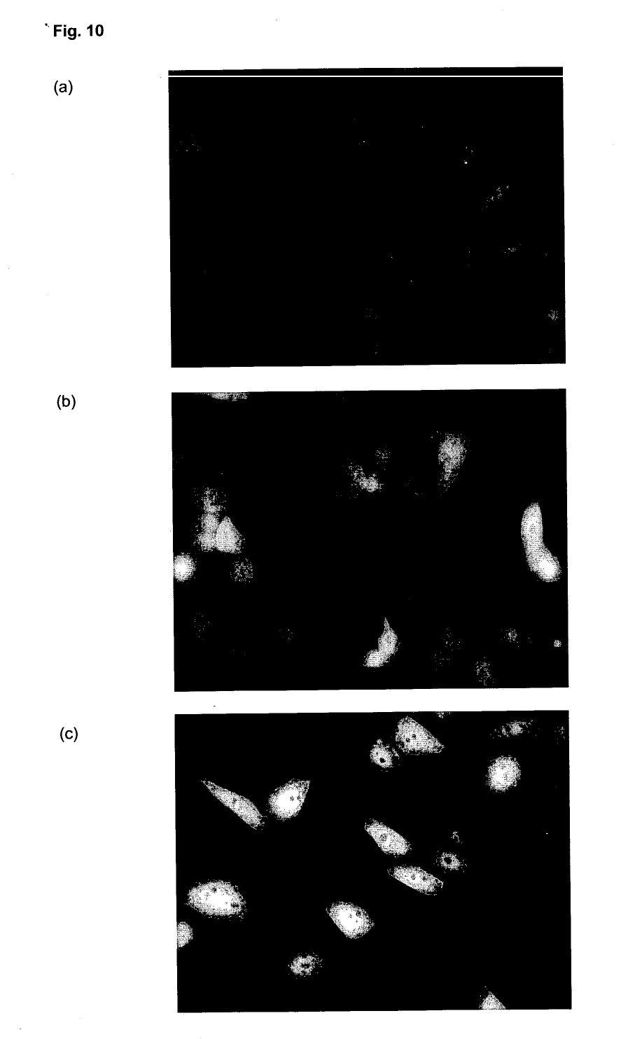

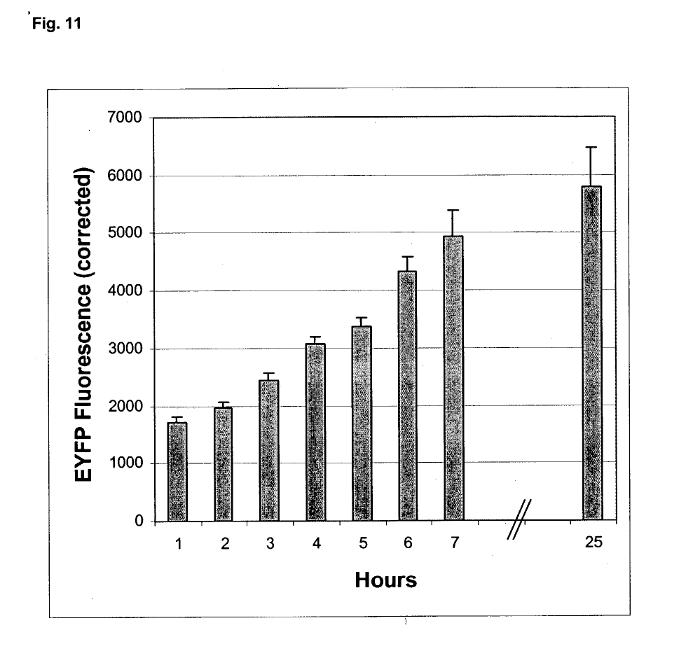

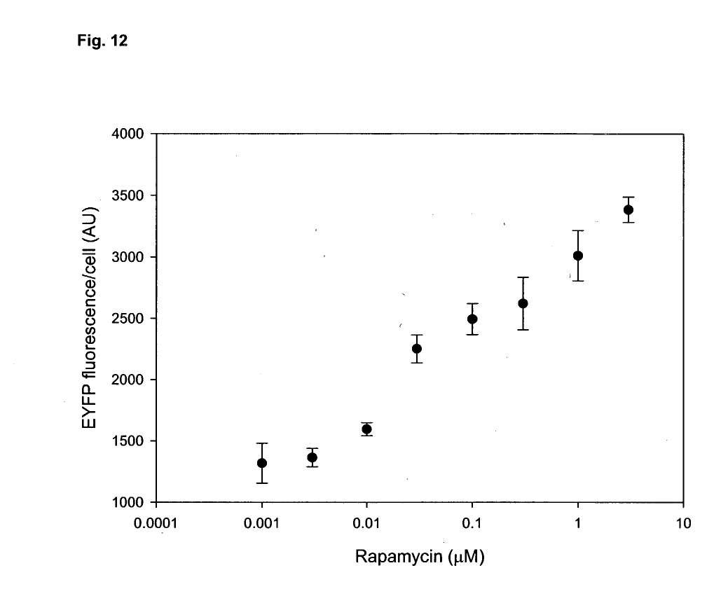

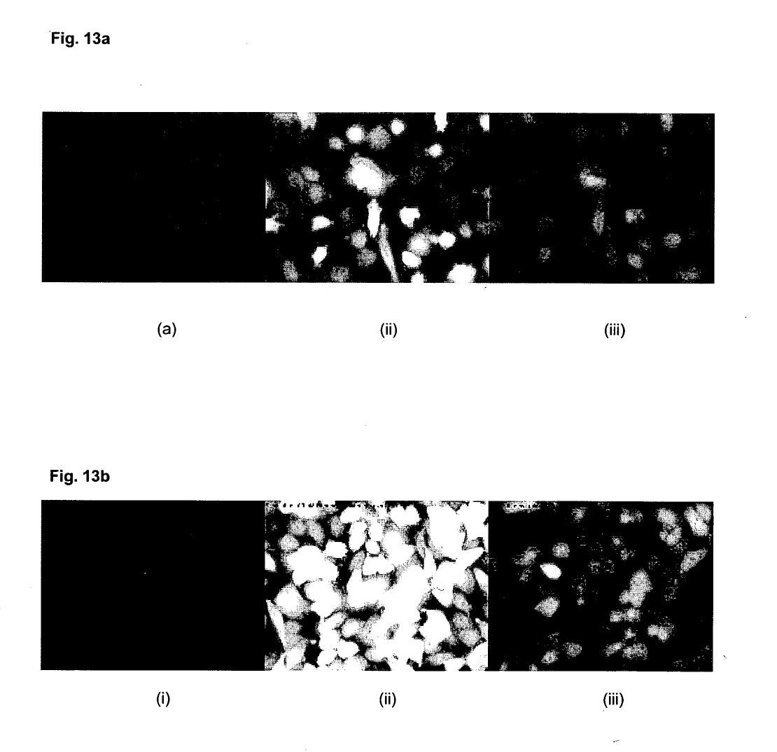

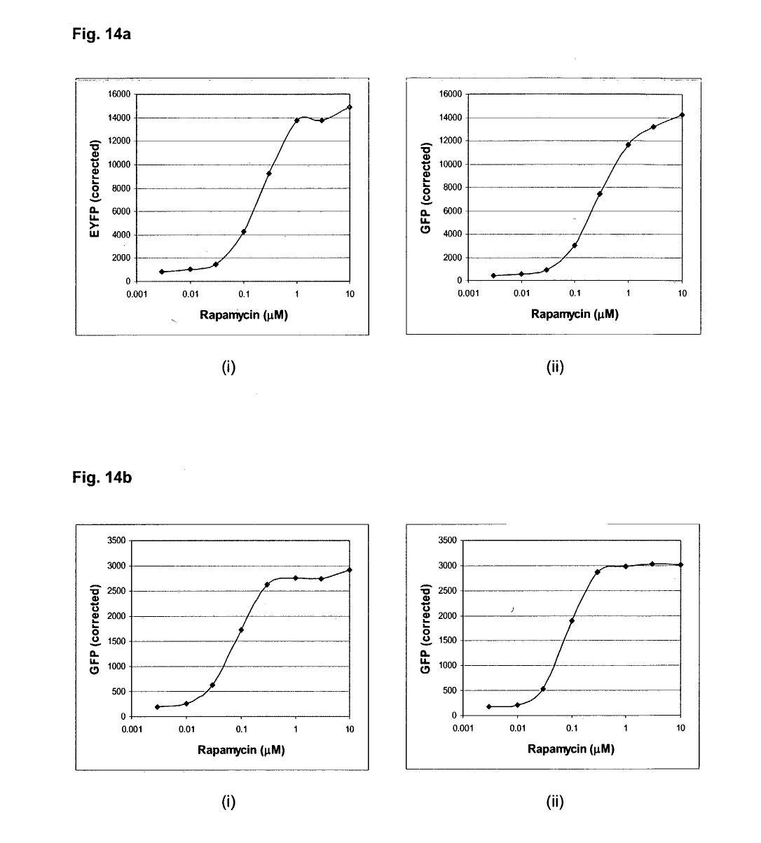

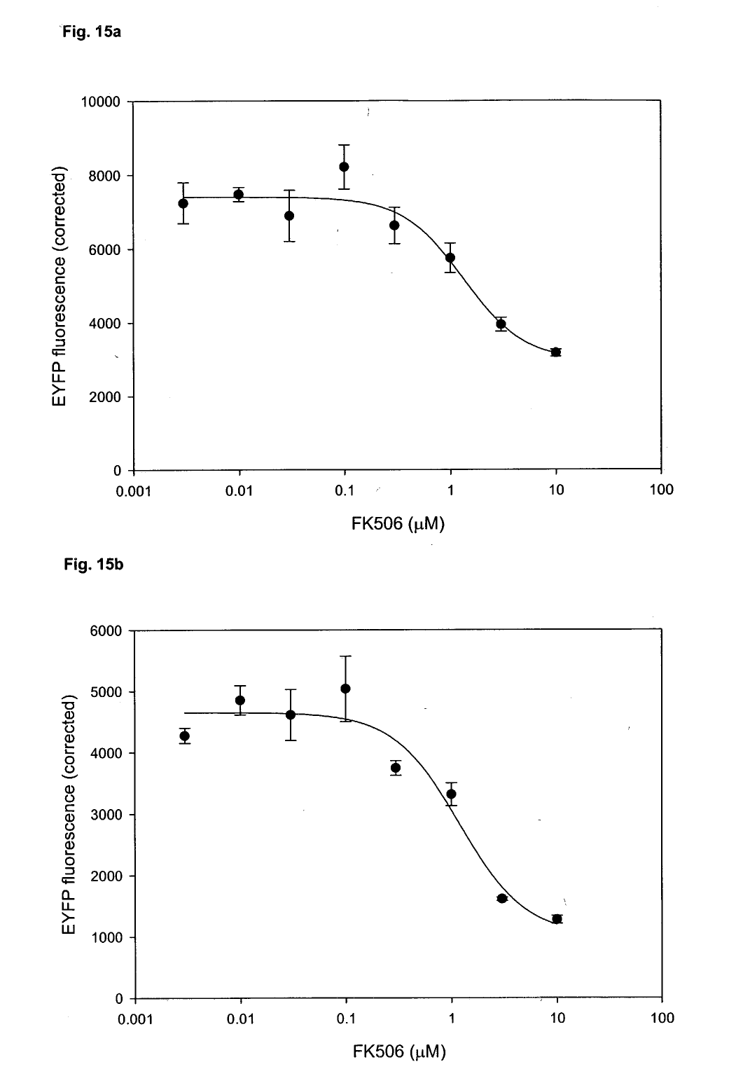

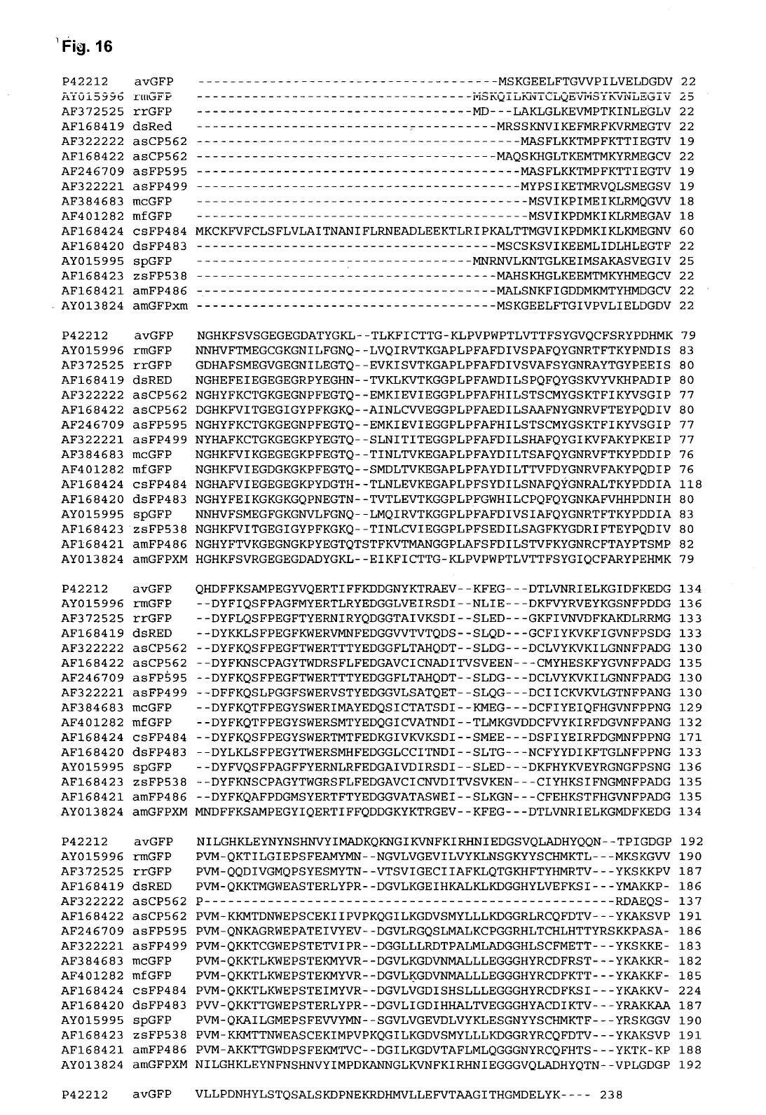

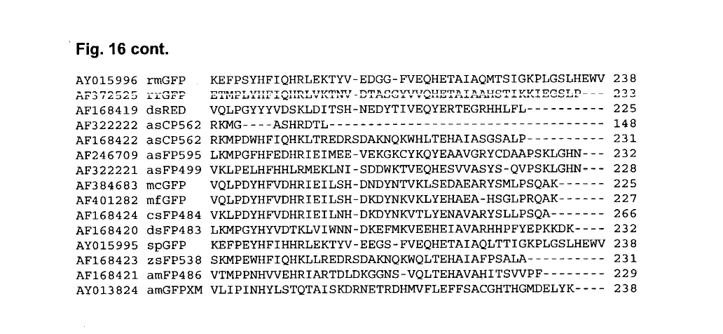

The same 16 bit images of fluorescent CHO-hIR cells co-transfected with NtermEGFP-NZ and CZ-CtermEGFP expression vectors or transfected with pEGFP-C1 as shown in FIG. 2 but the images are now shown with the same intensity scaling to allow comparison of fluorescence intensities. The cells that are transfected with complementation constructs that are based on a split between residues 172 and 173 (middle row) are clearly more fluorescent than the cells that are transfected with complementation constructs that are based on a split between residues 157 and 158 (top row). However, the cells transfected with the pEGFP-C1 construct (bottom row) show significantly stronger fluorescence at day 2.

FIG. 4

The unmanipulated microscope images shown in FIG. 3 were analysed using the ImageJ software package and data analysis was performed in Microsoft Excel. For each 16-bit monochrome IP Lab microscope image, pixel intensity data were produced in ImageJ and exported to an Excel spread-sheet for data analysis. The darkest and brightest 0.5% of the pixels were identified in each image and the average intensities of these two groups of pixels were calculated. The average intensity of the 0.5% darkest pixels was defined as the back ground fluorescence intensity (shown as white bars in the histogram) and the intensity of the 0.5% brightest pixels was defined as the maximum intensity. The difference in intensity between the maximum intensity and the background intensity was defined as the response (shown as cross hatched bars in the histogram). The sum of the background intensity and the response is equal to the maximum intensity. From the figure, it is clear that EGFP based fluorescence complementation using a split between residues 172 and 173, and probably anywhere else in this loop, is greatly superior to EGFP based fluorescence complementation using a split between residues 157 and 158 and probably also to splits anywhere else in this loop.

FIG. 5

Positions of appropriate fluorescent protein splitting sites are shown on ribbon and wire frame representations of GFP. The two representations show the same sites from two sides (molecule rotated approximately 180 degrees around a vertical axis).

FIG. 6

Co-transfection of expression vectors expressing EGFP and EYFP based complementation fragments as described in FIG. 3 to compare the abilities of the various complementation fragments to combine in cells and produce functional complexes. All images are scaled identically to allow direct comparison of fluorescence intensities between the images.

Single transfections with N-terminal fragments only resulted in no detectable fluorescence above the background level (data not shown). These N-terminal fragments contain amino acid residues 65-67 forming the chromophore in full-length GFP.

FIG. 7

Quantitative analysis of the images shown in FIG. 6. The results are in accord with the impressions from visual inspection of the cells. The data were produced as described in the legend to FIG. 4.

FIG. 8

Co-transfection of expression vectors expressing EGFP and EYFP based complementation fragments as described in FIG. 3 to compare the effects of mixing differently colored EGFP, EYFP and EYFP F64L fragments and to determine the influence of overlapping fragments, e.g. combining fragments encoding residues 1-172 and 158-238. All color combinations complement but typically less efficiently than in the correct combinations, i.e. when no or few residues overlap. Fragments having overlapping regions are also functional and this may be advantageous in experiments where longer linker sequences are or may be required by the fusion partners due to steric hindrance. This was not the case in this experiments where the fusion partners are leucine zippers. In the example (middle column), residues 158-172 were present in both fragments. In all situations, the F64L has a favorable effect on the fluorescence intensities. All images are scaled identically to allow direct comparison of fluorescence intensities between the images.

FIG. 9

Quantitative analysis of the images shown in FIG. 8. The results can be compared directly with the results shown in FIG. 7 and they are in accord with the impressions from visual inspection of the cells. The data were produced as described in the legend to FIG. 4.

FIG. 10

CHO-hIR [PS1767+PS1768] cells at 3 time points after treatment with 1 μM rapamycin. Note that image (c) was taken at 25 msec exposure, the previous 2 images at exposures of 100 msec each.

(a) is the starting condition for these cells (t=0), and fluorescence is barely visible in most cells, although it was noted that some cells (<5%) in the population had low, but appreciable, fluorescence before treatment.

(b), after 4 hours many cells (approximately 40%) had developed significantly greater EYFP fluorescence throughout the cytoplasmic and nuclear compartments.

(c) after 16 hours the response per cell had increased further and encompassed a larger proportion of the cell population (approximately 70%).

FIG. 11

The rate of development of cellular EYFP fluorescence following rapamycin treatment of the CHO-hIR [PS1767+PS1768] line. Cells were treated in 96-well plates with 3 μM rapamycin and the fluorescence measured. Treatment and measurements were made with the cells growing in HAM's medium+10% FBS, and fluorescence measurements were corrected for the background fluorescence from this medium. The graph demonstrates that the half-time for development of fluorescence is approximately 5 hours. Values corrected for HAM's background, each value a mean+sd for 8 measurements.

FIG. 12

Response curve to different rapamycin doses for the CHO-hIR [PS1769+PS1771] cell line. Cells were cultured in 96-well plates, treated with various rapamycin doses for 16 hours, then fixed and stained with Hoechst prior to determination of EYFP fluorescence/cell (arbitrary units) on the Ascent plate reader. Values are corrected for PBS background as well as cell number. The cell line shows approximately a 3-fold increase in the EYFP intensity/cell over the dose range of rapamycin used in this experiment.

FIG. 13

Each of the cell lines were fluorescence activated cell sorted (FACS) into 3 groups: (i) most green group (ii) medium to low-green group and (iii) black group. The ‘most green’ was discarded in each case, while the other 2 groups were cultured for further use.

A: CHO-hIR [ps1768+ps1767] FACS group ‘Black’ before stimulation (i), and after 16 hours stimulation with 100 nM rapamycin (ii) & (iii). Images (i) and (ii) were exposed for 100 msec, image (iii) for 25 msec.

B: CHO-hIR [ps1768+ps1767] FACS group ‘medium-low green’ before stimulation (i), and after 16 hours stimulation with 100 nM rapamycin (ii) & (iii). Images (i) and (ii) were exposed for 100 msec, image (iii) for 25 msec.

FIG. 14

Show the response of the ‘medium to low-green’ (a) and ‘black’ (b) FACS groups (respectively) derived from the CHO-hIR [PS1767+PS1768] parent line (see FIG. 13). Dose response to rapamycin was measured after 7 hours (i) and 30 hours (ii) for each cell line. Values for fluorescence have been corrected for plate & medium background. Increase in EYFP fluorescence is better than, 20-fold the unstimulated value in each case.

FIG. 15

Show dose-response competition curves for FK506 versus 100 nM rapamycin in two of the FACS'd lines, (a) CHO-hIR [PS1768+PS1767] ‘mid to low-green’ group, and (b) CHO-hIR [PS1769+PS1771] ‘black’ group. EC₅₀ values in both cases are approximately 1.2 μM FK506. The cells were incubated overnight (16 hours) with mixtures of the two compounds, then fixed and stained with Hoechst prior to determination of EYFP fluorescence/cell on an Ascent plate reader. Plate and solution backgrounds have been subtracted; the dashed lines on each graph indicate the prestimulated fluorescence levels for each cell line in these experiments.

FIG. 16

Alignment of fluorescent proteins.

### TABLES

TABLE 2

Oligo nucleotides used in cloning. Oligo nucleotides beginning with
+UZ,uz,1/43 P* are phosphorylated at the 5′ end to permit ligation.

| | | |
|---|---|---|
| Oligo | | SEQ |
| nucleo | | ID |
| -tide | Oligo nucleotide sequence (5′ end to 3′ end) | NO: |

| | | | |
|---|---|---|---|
| 1268 | ATTB2- CCTACTGCTTTGAGATTCGTCGG | 15 | |
| 1272 | ATTB2- GTCATTCCAGTTTTAGAAGCTC | 16 | |
| 1282 | CAGACAATCTGTGTGGGCACTCGACCGG | 17 | |
| 2110 | P*CATGGCCGGTGGTACCGGTTCCGGTGCCCTGAAGAAGGAGCTGCAGG | 18 | |
| 2111 | P*AGCTCCTTCTTCAGGGCACCGGAACCGGTACCACCGGC | 19 | |
| 2112 | P*CCAACAAGAAGGAGCTGGCCCAGCTGAAGTGGGAGCTGCAG | 20 | |
| 2113 | P*CTCCCACTTCAGCTGGGCCAGCTCCTTCTTGTTGGCCTGC | 21 | |
| 2114 | P*GCCCTGAAGAAGGAGCTGGCCCAGTAG | 22 | |
| 2115 | P*GATCCTACTGGGCCAGCTCCTTCTTCAGGGCCTGCAG | 23 | |
| 2116 | P*CATGGCCAGCGAGCAGCTGGAGAAGAAGCTGCAGGCCCTG | 24 | |
| 2117 | P*CCTGCAGCTTCTTCTCCAGCTGCTCGCTGGC | 25 | |
| 2118 | p*GAGAAGAAGCTGGCCCAGCTGGAGTGGAAGAACCAGGCCCTGGAG | 26 | |
| 2119 | P*GGCCTGGTTCTTCCACTCCAGCTGGGCCAGCTTCTTCTCCAGGG | 27 | |
| 2120 | P*AAGAAGCTGGCCCAGGGCGGCACCGGTTAG | 28 | 28 |
| 2121 | P*GATCCTAACCGGTGCCGCCCTGGGCCAGCTTCTTCTCCAG | 29 | |
| 2128 | GGCGCCATGGTGAGCAAGGGCGAG | 30 | |
| 2129 | GCCGGACCGGTACCACCGTTGTACTCCAGCTTGTG | 31 | |
| 2130 | GCCGGACCGGTACCACCCTGCTTGTCGGCCATG | 32 | |
| 2131 | GCCGGACCGGTACCACCCTCGATGTTGTGGCGGATC | 33 | |
| 2132 | CCCCGGATCCTACTTGTACAGCTCGTCCATGC | 34 | |
| 2133 | GGCGCCATGGGCACCGGTTACAACAGCCACAACGTC | 35 | |
| 2134 | GGCGCCATGGGCACCGGTAAGAACGGCATCAAGGTG | 36 | |
| 2135 | GGCGCCATGGGCACCGGTGACGGCAGCGTGCAGCTC | 37 | |
| 2219 | GGGGGCTAGCGCCACCATGGTGAGCAAGGGCGAG | 38 | |
| 2222 | GCGCGGGATCCGATATCGCCAGAGCCAGAGCCAGAGCCCTCGATGTTGTGGCGGATC | 39 | |
| 2225 | GGGGGCTAGCGATATCCGGCTCTGGCTCTGGCTCTGGCGACGGCAGCGTGCAGCTC | 40 | |
| 2333 | GCCCACCCTCGTGACCACCTTCGGCTACGGCCTGCAGTGCTTCGCCCGCTACCCCGACC | 41 | |
| | ACATG | | |
| 2334 | CATGTGGTCGGGGTAGCGGGCGAAGCACTGCAGGCCGTAGCCGAAGGTGGTCACGAGGG | 42 | |
| | TGGGC | | |
| 2335 | GCCCACCCTCGTGACCACCCTGGGCTACGGCCTGCAGTGCTTCGCCCGCTACCCCGACC | 43 | |
| | ACATG | | |
| 2336 | CATGTGGTCGGGGTAGCGGGCGAAGCACTGCAGGCCGTAGCCCAGGGTGGTCACGAGGG | 44 | |
| | TGGGC | | |
| 2337 | GACAACCACTACCTGAGCTACCAGTCCGCCCTGAGC | 45 | |
| 2338 | GCTCAGGGCGGACTGGTAGCTCAGGTAGTGGTTGTC | 46 | |
| 2442 | ATTB1- CCACCATGGGAGTGCAGGTGGAAACC | 47 | |
| 2443 | ATTB2- CTTCCAGTTTTAGAAGCTC | 48 | |
| 2444 | ATTB1- CCACCATGGAGATGTGGCATGAAGGCCTG | 49 | |
| 2445 | ATTB2- CCTGCTTTGAGATTCGTCGGAACAC | 50 | |
| 9655 | TCCTAGGTCAGTCCTGCTCCTCGGCCACGAAGTGCAC | 51 | |
| | TCCTAGGCTGCAGCACGTGTTGACAATTAATCATCGG | | |
| 9658 | CAGACAATCTGTGTGGGCACTCGACCGG | 52 | |

TABLE 3
Primer pairs used in EGFP fragment amplification

| Protein encoded by PCR fragment | 5′ primer | 3′ primer |
|---|---|---|
| EGFP(1-144) | 2128 | 2129 |
| EGFP(1-157) | 2128 | 2130 |
| EGFP(1-172) | 2128 | 2131 |
| EGFP(145-238) | 2133 | 2132 |
| EGFP(158-238) | 2134 | 2132 |
| EGFP(173-238) | 2135 | 2132 |

TABLE 4
Cloning and expression vectors

| | | | Selection |
|---|---|---|---|
| Vector | Expressed protein | Promotor | E. coli/mamm. |
| pEGFP-C1 | EGFP | CMV | kan/neo |
| PS0609 | EGFP | CMV | zeo/zeo |
| pTrcHis-A | no insert | Trc | amp/none |
| PS1515 | NZ leucine zipper | Trc | amp/none |
| PS1516 | CZ leucine zipper | Trc | amp/none |
| PS1614 | NtermEGFP144-NZ | Trc | amp/none |
| PS1596 | NtermEGFP157-NZ | Trc | amp/none |
| PS1597 | NtermEGFP172-NZ | Trc | amp/none |
| PS1615 | CZ-CtermEGFP145 | Trc | amp/none |
| PS1594 | CZ-CtermEGFP158 | Trc | amp/none |
| PS1595 | CZ-CtermEGFP173 | Trc | amp/none |
| PS1559 | NtermEGFP157-NZ | CMV | kan/neo |
| PS1560 | NtermEGFP172-NZ | CMV | kan/neo |
| PS1557 | CZ-CtermEGFP158 | CMV | zeo/zeo |
| PS1558 | CZ-CtermEGFP173 | CMV | zeo/zeo |
| PS1639 | NtermEYFP157-NZ | CMV | kan/neo |
| PS1642 | NtermEYFP172-NZ | CMV | kan/neo |
| PS1640 | NtermE[F64L]157YFP-NZ | CMV | kan/neo |
| PS1641 | NtermE[F64L]YFP172-NZ | CMV | kan/neo |
| PS1637 | CZ-CtermEYFP158 | CMV | zeo/zeo |
| PS1638 | CZ-CtermEYFP173 | CMV | zeo/zeo |
| PS1769 | NtermE[F64L]YFP172-FKBP | CMV | kan/neo |
| PS1767 | NtermE[F64L]YFP172-FRB | CMV | kan/neo |
| PS1771 | FRB-CtermEYPF173 | CMV | zeo/zeo |
| PS1768 | FKBP-CtermEYFP173 | CMV | zeo/zeo |

TABLE 5
Sequence names and numbers

| SEQ ID NO: | Name |
|---|---|
| 1 | Amino acid sequence of GFP |
| 2 | Amino acid sequence of GFP Y66W |
| 3 | Amino acid sequence of GFP Y66H |
| 4 | Amino acid sequence of EGFP |
| 5 | Amino acid sequence of EYFP |
| 6 | Amino acid sequence of EYFP F64L variant |
| 7 | Nucleic acid sequence of NZ |
| 8 | Amino acid sequence of NZ |
| 9 | Nucleic acid sequence of CZ |
| 10 | Amino acid sequence of CZ |
| 11 | NtermE[F64L]YFP172 and FKBP linker sequence |
| 12 | NtermE[F64L]YFP172 and FRB linker sequence |
| 13 | FRB and CtermEYFP173 linker sequence |
| 14 | FKBP and CtermEYFP173 linker sequence |
| 15-52 | Primer sequence (see Table 2) |

All cited patens, publications, copending applications, and provisional applications referred to in this application are herein incorporated by reference.

The invention being thus described, it will be obvious that the same may be varied in many ways. Such avariations are not to be regarded as a departure from the spirit and scope of the present inventions, and all such modifications as would be obvious to one skilled in the art are intended to be included within the scope of the following claims.

1. Two GFP fragments comprising
(a) an N-terminal fragment of GFP, comprising a continuous stretch of amino acids from amino acid number 1 to amino acid number X of GFP, wherein the peptide bond between amino acid number X and amino acid number X+1 is within a loop of GFP and
(b) a C-terminal fragment of GFP, comprising a continuous stretch of amino acids from amino acid number X+1 to amino acid number 238 of GFP.

2. Two GFP fragments comprising
(a) an N-terminal fragment of GFP, comprising a continuous stretch of amino acids from amino acid number 1 to amino acid number X of GFP, wherein the peptide bond between amino acid number X and amino acid number X+1 is within a loop of GFP and
(b) a C-terminal fragment of GFP, comprising a continuous stretch of amino acids from amino acid number Y+1 to amino acid number 238 of GFP, wherein Y<X creating an overlap of the two GFP fragments, and wherein the peptide bond between amino acid Y and amino acid Y+1 is within a loop of GFP.

3. Two GFP fragments according to any of the preceding claims, wherein GFP is selected from the group consisting of EGFP, EYFP, ECFP, dsRed and Renilla GFP.

4. Two GFP fragments according to any of the preceding claims, wherein the GFP is EGFP.

5. Two GFP fragments according to any of the preceding claims, wherein the GFP is EYFP.

6. Two GFP fragments according to any of the preceding claims, wherein the amino acid in position 1 preceding the chromophore has been mutated to provide an increase of fluorescence intensity.

7. Two GFP fragments according to the preceding claim, wherein the amino acid F in position 1 preceding the chromophore has been substituted by L.

8. Two GFP fragments according to any of the preceding claims, wherein the GFP has been mutated to further contain the S72A mutation.

9. Two GFP fragments according to any of the preceding claims, wherein X is between 9 and 10 within the Thr9-Val11 loop; or between 23 and 24 within the Asn23-His25 loop; or between 38 and 39 within the Thr38-Gly40 loop; or between 48 and 55 within the Cys48-Pro56 loop; or between 72 and 75 within the Ser72-Asp76 loop; or between 81 and 82 within the His81-Phe83 loop; or between 88 and 89 within the Met88-Glu90 loop; between 101 and 102 within the Lys101-Asp103 loop; or between 114 and 117 within the Phe114-Thr118 loop; or between 128 and 144 within the Ile 128-Tyr145 loop; or between 154 and 159 within the Ala154-Gly160 loop; or between 171 and 174 within the Ile171-Ser175 loop; or between 188 and 196 within the Ile188-Asp197 loop; or between 210 and 214 within the Asp210-Art215 loop.

10. Two GFP fragments according to the preceding claim, wherein X is between 154 and 159 within the Ala154-Gly160 loop.

11. Two GFP fragments according to the preceding claim, wherein X is 157 within the Ala154-Gly160 loop.

12. Two GFP fragments according to the preceding claim, wherein X is between 171 and 174 within the Ile171-Ser175 loop.

13. Two GFP fragments according to any of the preceding claims, wherein X is 172 within in Ile171-Ser175 loop.

14. Two GFP fragments according to the preceding claim, wherein Y is between 154 and 159 within the Ala154-Gly160 loop.

15. Two GFP fragments according to the preceding claim, wherein Y is 157 within the Ala154-Gly160 loop.

16. Two GFP fragments according to any of the preceding claims, wherein X is 172 within in Ile171-Ser175 loop and wherein Y is 157 within the Ala154-Gly160 loop.

17. Two GFP fragments according to any of the preceding claims, wherein the N-terminal fragment of GFP is fused in frame with a first protein of interest.

18. Two GFP fragments according to any of the preceding claims, wherein the first protein of interest is fused to the N-terminal of the N-terminal fragment of GFP

19. Two GFP fragments according to any of the preceding claims, wherein the first protein of interest is fused to the C-terminal of the N-terminal fragment of GFP.

20. Two GFP fragments according to any of the preceding claims, wherein the C-terminal fragment of GFP is fused in frame with a second protein of interest.

21. Two GFP fragments according to any of the preceding claims, wherein the second protein of interest is fused to the N-terminal of the C-terminal fragment of GFP.

22. Two GFP fragments according to any of the preceding claims, wherein the second protein of interest is fused to the C-terminal of the C-terminal fragment of GFP.

23. Two GFP fragments according to any of the preceding claims, wherein the N-terminal fragment of GFP fused in frame to a first protein of interest further comprises a linker sequence between the N-terminal fragment of GFP and the first protein of interest.

24. Two GFP fragments according to any of the preceding claims, wherein the C-terminal fragment of GFP fused in frame to a second protein of interest further comprises a linker sequence between the C-terminal fragment of GFP and the second protein of interest.

25. Two GFP fragments according to any of the preceding claims, wherein the GFP is EYFP further containing an F64L mutation, wherein X is 172, wherein the first protein of interest fused to the N-terminal fragment of GFP is fused to the C-terminal of the N-terminal fragment of GFP and wherein the second protein of interest fused to the C-terminal fragment of GFP is fused to the N-terminal of the C-terminal fragment of GFP.

26. Two GFP fragments according to any of the preceding claims, wherein the GFP is EYFP further containing an F64L mutation, wherein X is 157, wherein the first protein of interest fused to the N-terminal fragment of GFP is fused to the C-terminal of the N-terminal fragment of GFP and wherein the second protein of interest fused to the C-terminal fragment of GFP is fused to the N-terminal of the C-terminal fragment of GFP.

27. The N-terminal fragment of GFP according to any of the preceding claims.

28. The C-terminal fragment of GFP according to any of the preceding claims.

29. Nucleic acid encoding a fragment according to any of the preceding claims.

30. A cell comprising an N-terminal fragment of GFP according to any of the preceding claims.

31. A cell comprising a C-terminal fragment of GFP according to any of the preceding claims.

32. A cell comprising the two GFP fragments according to any of the preceding claims.

33. A vector comprising the two GFP fragments according to any of the preceding claims.

34. A vector comprising the N-terminal fragment of GFP according to any of the preceding claims.

35. A vector comprising the C-terminal fragment of GFP according to any of the preceding claims.

36. A plasmid comprising the two GFP fragments according to any of the preceding claims.

37. A plasmid comprising the N-terminal fragment of GFP according to any of the preceding claims.

38. A plasmid comprising the C-terminal fragment of GFP according to any of the preceding claims.

39. A method for detecting the interaction between two proteins of interest comprising the steps of:
(a) providing at least one cell that contains two heterologous conjugates,
the first heterologous conjugate comprising a first protein of interest conjugated to an N-terminal fragment of GFP according to any of the preceding claims,
the second heterologous conjugate comprising a second protein of interest conjugated to a C-terminal fragment of GFP according to any of the preceding claims; and
b) measuring the fluorescence from the at least one cell,
fluorescent cells indicating interaction between the two proteins of interest.

40. A method for monitoring the interaction between two proteins of interest comprising the steps of:
(a) providing at least one cell containing at least one stretch of nucleic acid encoding for two heterologous conjugates, the first heterologous conjugate comprising a first protein of interest conjugated to an N-terminal fragment of GFP according to any of the preceding claims, the second heterologous conjugate comprising a second protein of interest conjugated to a C-terminal fragment of GFP according to any of the preceding claims;
(b) culturing the at least one cell under conditions allowing expression; and
(c) measuring the fluorescence from the at least one cell,
fluorescent cells indicating interaction between the two proteins of interest.

41. A method according to any of the preceding claims for detecting new interaction partners, wherein one of the proteins of interest is known, and the other protein of interest is an unknown protein comprising the additional step of
parallel transfection of the cells with both heterologous conjugates,
cells expressing interaction partners to the know protein of interest will be fluorescent and thereby easily detectable.

42. A method according to any of the preceding claims for detecting new interaction partners, wherein one of the proteins of interest is known, and the other protein of interest is an unknown protein comprising the additional steps of
establishing a cell line that stabilly expresses the heterologous conjugate comprising the known protein of interest;
transfecting said cell line with a library of heterologous conjugates comprising the potential interaction partners;
cells expressing interaction partners to the know protein of interest will be fluorescent and thereby easily detectable.

43. A method for detecting compounds that induce interaction between two proteins of interest comprising the steps of:
(a) providing at least one cell that contains two heterologous conjugates,
the first heterologous conjugate comprising a first protein of interest conjugated to an N-terminal fragment of GFP as described above,
the second heterologous conjugate comprising a second protein of interest conjugated to a C-terminal fragment of GFP as described above; and
(b) measuring the fluorescence from the at least one cell of step (a),
(c) apply a test compound to the at least one cell of step (b)
(d) measuring the fluorescence from the at least one cell of step (c);
an increase in fluorescence observed from step (b) to step (d) indicating that the test compound added in step (c) is capable of inducing interaction between the two proteins of interest.

44. A method for screening for compounds that interfere with a conditional interaction between two protein components comprising the steps of:
(a) providing at least one cell that contains two heterologous conjugates,
the first heterologous conjugate comprising a first protein of interest conjugated to an N-terminal fragment of GFP as described above,
the second heterologous conjugate comprising a second protein of interest conjugated to a C-terminal fragment of GFP as described above; and
(b) measuring the fluorescence from the at least one cell of step (a),
(c) apply a test compound and a compound that induces interaction between two proteins of interest to the at least one cell of step (b)
(d) measuring the fluorescence from the at least one cell of step (c);
an increase in fluorescence observed from step (b) to step (d) indicating that the test compound added in step (c) does not prevent interaction between the two proteins of interest; whereas a lesser increase in fluorescence observed from step (b) to step (d) indicates that the test compound will interfere with the induced interaction between the two proteins of interest.

45. A method according to any of the preceding claims wherein the at least one cell is a heterogeneous cell population, comprising the additional steps of
removal of the most green cells;
removal of the black cells;
hereby obtaining “medium to low-green” cells.

46. A method according to the preceding claim, wherein the removal steps are carried out by FACS.

47. A method according to any of the preceding claims wherein the at least one cell is a heterogeneous cell population with a high dynamic range, comprising the additional steps of:
stimulating the “medium to low-green” cells with a compound that induces interaction between two proteins of interest and;
allow sufficient time to pass to let the proteins interact and the fluorescent protein fragments fold and become fluorescent;
isolate the most green cells;
this population of cells will have a very low background and still be capable of forming the fluorescent protein upon interaction between the two proteins of interest.

48. A method according to the preceding claim, wherein the isolation step is carried out by FACS.

49. A method according to any of the preceding claims, wherein the at least one cell is a mammalian cell.


SEQUENCE LISTING (71 sequences)

SEQ ID NO: 1 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 2 - protein, 238 aa, Aequorea victoria
MSKGEELFTGVVPILVELDGDVNGHKFSVSGEGEGDATYGKLTLKFICTTGKLPVPWPTLVTTFSWGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 3 - protein, 238 aa, Aequorea victoria
MSKGEELFTGVVPILVELDGDVNGHKFSVSGEGEGDATYGKLTLKFICTTGKLPVPWPTLVTTFSHGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 4 - protein, 239 aa, Aequorea victoria
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 5 - protein, 239 aa, Aequorea victoria
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 6 - protein, 239 aa, Aequorea victoria
MVSKGEELFTGVVPILVELDGDVNGHKFSVSGEGEGDATYGKLTLKFICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 7 - DNA, 121 bp, Artificial Sequence
  CDS (3)..(116): Constructed sequence
ccatggccggtggtaccggttccggtgccctgaagaaggagctgcaggccaacaagaaggagctggcccagctgaagtgggagctgcaggccctgaagaaggagctggcccagtaggatcc

SEQ ID NO: 8 - protein, 37 aa, Artificial Sequence
  Constructed sequence
MAGGTGSGALKKELQANKKELAQLKWELQALKKELAQ

SEQ ID NO: 9 - DNA, 121 bp, Artificial Sequence
  CDS (3)..(116): Constructed sequence
ccatggccagcgagcagctggagaagaagctgcaggccctggagaagaagctggcccagctggagtggaagaaccaggccctggagaagaagctggcccagggcggcaccggttaggatcc

SEQ ID NO: 10 - protein, 37 aa, Artificial Sequence
  Constructed sequence
MASEQLEKKLQALEKKLAQLEWKNQALEKKLAQGGTG

SEQ ID NO: 11 - protein, 19 aa, Artificial Sequence
  Constructed sequence
GSGSGSGDITSLYKKAGST

SEQ ID NO: 12 - protein, 19 aa, Artificial Sequence
  Constructed sequence
GSGSGSGDITSLYKKAGST

SEQ ID NO: 13 - protein, 18 aa, Artificial Sequence
  Constructed sequence
DPAFLYKVVISGSGSGSG

SEQ ID NO: 14 - protein, 18 aa, Artificial Sequence
  Constructed sequence
DPAFLYKVVISGSGSGSG

SEQ ID NO: 15 - DNA, 23 bp, Artificial Sequence
  misc_feature: Primer sequence
cctactgctttgagattcgtcgg

SEQ ID NO: 16 - DNA, 22 bp, Artificial Sequence
  misc_feature: Primer sequence
gtcattccagttttagaagctc

SEQ ID NO: 17 - DNA, 28 bp, Artificial Sequence
  misc_feature: Primer sequence
cagacaatctgtgtgggcactcgaccgg

SEQ ID NO: 18 - DNA, 47 bp, Artificial Sequence
  misc_feature: Primer sequence
catggccggtggtaccggttccggtgccctgaagaaggagctgcagg

SEQ ID NO: 19 - DNA, 38 bp, Artificial Sequence
  misc_feature: Primer sequence
agctccttcttcagggcaccggaaccggtaccaccggc

SEQ ID NO: 20 - DNA, 41 bp, Artificial Sequence
  misc_feature: Primer sequence
ccaacaagaaggagctggcccagctgaagtgggagctgcag

SEQ ID NO: 21 - DNA, 40 bp, Artificial Sequence
  misc_feature: Primer sequence
ctcccacttcagctgggccagctccttcttgttggcctgc

SEQ ID NO: 22 - DNA, 27 bp, Artificial Sequence
  misc_feature: Primer sequence
gccctgaagaaggagctggcccagtag

SEQ ID NO: 23 - DNA, 37 bp, Artificial Sequence
  misc_feature: Primer sequence
gatcctactgggccagctccttcttcagggcctgcag

SEQ ID NO: 24 - DNA, 40 bp, Artificial Sequence
  misc_feature: Primer sequence
catggccagcgagcagctggagaagaagctgcaggccctg

SEQ ID NO: 25 - DNA, 31 bp, Artificial Sequence
  misc_feature: Primer sequence
cctgcagcttcttctccagctgctcgctggc

SEQ ID NO: 26 - DNA, 45 bp, Artificial Sequence
  misc_feature: Primer sequence
gagaagaagctggcccagctggagtggaagaaccaggccctggag

SEQ ID NO: 27 - DNA, 44 bp, Artificial Sequence
  misc_feature: Primer sequence
ggcctggttcttccactccagctgggccagcttcttctccaggg

SEQ ID NO: 28 - DNA, 30 bp, Artificial Sequence
  misc_feature: Primer sequence
aagaagctggcccagggcggcaccggttag

SEQ ID NO: 29 - DNA, 40 bp, Artificial Sequence
  misc_feature: Primer sequence
gatcctaaccggtgccgccctgggccagcttcttctccag

SEQ ID NO: 30 - DNA, 24 bp, Artificial Sequence
  misc_feature: Primer sequence
ggcgccatggtgagcaagggcgag

SEQ ID NO: 31 - DNA, 35 bp, Artificial Sequence
  misc_feature: Primer sequence
gccggaccggtaccaccgttgtactccagcttgtg

SEQ ID NO: 32 - DNA, 33 bp, Artificial Sequence
  misc_feature: Primer sequence
gccggaccggtaccaccctgcttgtcggccatg

SEQ ID NO: 33 - DNA, 36 bp, Artificial Sequence
  misc_feature: Primer sequence
gccggaccggtaccaccctcgatgttgtggcggatc

SEQ ID NO: 34 - DNA, 32 bp, Artificial Sequence
  misc_feature: Primer sequence
ccccggatcctacttgtacagctcgtccatgc

SEQ ID NO: 35 - DNA, 36 bp, Artificial Sequence
  misc_feature: Primer sequence
ggcgccatgggcaccggttacaacagccacaacgtc

SEQ ID NO: 36 - DNA, 36 bp, Artificial Sequence
  misc_feature: Primer sequence
ggcgccatgggcaccggtaagaacggcatcaaggtg

SEQ ID NO: 37 - DNA, 36 bp, Artificial Sequence
  misc_feature: Primer sequence
ggcgccatgggcaccggtgacggcagcgtgcagctc

SEQ ID NO: 38 - DNA, 34 bp, Artificial Sequence
  misc_feature: Primer sequence
gggggctagcgccaccatggtgagcaagggcgag

SEQ ID NO: 39 - DNA, 57 bp, Artificial Sequence
  misc_feature: Primer sequence
gcgggggatccgatatcgccagagccagagccagagccctcgatgttgtggcggatc

SEQ ID NO: 40 - DNA, 56 bp, Artificial Sequence
  misc_feature: Primer sequence
gggggctagcgatatccggctctggctctggctctggcgacggcagcgtgcagctc

SEQ ID NO: 41 - DNA, 64 bp, Artificial Sequence
  misc_feature: Primer sequence
gcccaccctcgtgaccaccttcggctacggcctgcagtgcttcgcccgctaccccgaccacatg

SEQ ID NO: 42 - DNA, 64 bp, Artificial Sequence
  misc_feature: Primer sequence
catgtggtcggggtagcgggcgaagcactgcaggccgtagccgaaggtggtcacgagggtgggc

SEQ ID NO: 43 - DNA, 64 bp, Artificial Sequence
  misc_feature: Primer sequence
gcccaccctcgtgaccaccctgggctacggcctgcagtgcttcgcccgctaccccgaccacatg

SEQ ID NO: 44 - DNA, 64 bp, Artificial Sequence
  misc_feature: Primer sequence
catgtggtcggggtagcgggcgaagcactgcaggccgtagcccagggtggtcacgagggtgggc

SEQ ID NO: 45 - DNA, 36 bp, Artificial Sequence
  misc_feature: Primer sequence
gacaaccactacctgagctaccagtccgccctgagc

SEQ ID NO: 46 - DNA, 36 bp, Artificial Sequence
  misc_feature: Primer sequence
gctcagggcggactggtagctcaggtagtggttgtc

SEQ ID NO: 47 - DNA, 26 bp, Artificial Sequence
  misc_feature: Primer sequence
ccaccatgggagtgcaggtggaaacc

SEQ ID NO: 48 - DNA, 19 bp, Artificial Sequence
  misc_feature: Primer sequence
cttccagttttagaagctc

SEQ ID NO: 49 - DNA, 29 bp, Artificial Sequence
  misc_feature: Primer sequence
ccaccatggagatgtggcatgaaggcctg

SEQ ID NO: 50 - DNA, 25 bp, Artificial Sequence
  misc_feature: Primer sequence
cctgctttgagattcgtcggaacac

SEQ ID NO: 51 - DNA, 74 bp, Artificial Sequence
  misc_feature: Primer sequence
tcctaggtcagtcctgctcctcggccacgaagtgcactcctaggctgcagcacgtgttgacaattaatcatcgg

SEQ ID NO: 52 - DNA, 28 bp, Artificial Sequence
  misc_feature: Primer sequence
cagacaatctgtgtgggcactcgaccgg

SEQ ID NO: 53 - protein, 238 aa, Aequorea Victoria; identical to SEQ ID NO: 1

SEQ ID NO: 54 - protein, 238 aa, Renilla muelleri
MSKQILKNTCLQEVMSYKVNLEGIVNNHVFTMEGCGKGNILFGNQLVQIRVTKGAPLPFAFDIVSPAFQYGNRTFTKYPNDISDYFIQSFPAGFMYERTLRYEDGGLVEIRSDINLIEDKFVYRVEYKGSNFPDDGPVMQKTILGIEPSFEAMYMNNGVLVGEVILVYKLNSGKYYSCHMKTLMKSKGVVKEFPSYHFIQHRLEKTYVEDGGFVEQHETAIAQMTSIGKPLGSLHEWV

SEQ ID NO: 55 - protein, 233 aa, Renilla reniformis
MDLAKLGLKEVMPTKINLEGLVGDHAFSMEGVGEGNILEGTQEVKISVTKGAPLPFAFDIVSVAFSYGNRAYTGYPEEISDYFLQSFPEGFTYERNIRYQDGGTAIVKSDISLEDGKFIVNVDFKAKDLRRMGPVMQQDIVGMQPSYESMYTNVTSVIGECIIAFKLQTGKHFTYHMRTVYKSKKPVETMPLYHFIQHRLVKTNVDTASGYVVQHETAIAAHSTIKKIEGSLP

SEQ ID NO: 56 - protein, 225 aa, Discosoma sp.
MRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 57 - protein, 148 aa, Anemonia sulcata
MASFLKKTMPFKTTIEGTVNGHYFKCTGKGEGNPFEGTQEMKIEVIEGGPLPFAFHILSTSCMYGSKTFIKYVSGIPDYFKQSFPEGFTWERTTTYEDGGFLTAHQDTSLDGDCLVYKVKILGNNFPADGPRDAEQSRKMGASHRDTL

SEQ ID NO: 58 - protein, 231 aa, Zoanthus sp.
MAQSKHGLTKEMTMKYRMEGCVDGHKFVITGEGIGYPFKGKQAINLCVVEGGPLPFAEDILSAAFNYGNRVFTEYPQDIVDYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALP

SEQ ID NO: 59 - protein, 232 aa, Anemonia sulcata
MASFLKKTMPFKTTIEGTVNGHYFKCTGKGEGNPFEGTQEMKIEVIEGGPLPFAFHILSTSCMYGSKTFIKYVSGIPDYFKQSFPEGFTWERTTTYEDGGFLTAHQDTSLDGDCLVYKVKILGNNFPADGPVMQNKAGRWEPATEIVYEVDGVLRGQSLMALKCPGGRHLTCHLHTTYRSKKPASALKMPGFHFEDHRIEIMEEVEKGKCYKQYEAAVGRYCDAAPSKLGHN

SEQ ID NO: 60 - protein, 228 aa, Anemonia sulcata
MYPSIKETMRVQLSMEGSVNYHAFKCTGKGEGKPYEGTQSLNITITEGGPLPFAFDILSHAFQYGIKVFAKYPKEIPDFFKQSLPGGFSWERVSTYEDGGVLSATQETSLQGDCIICKVKVLGTNFPANGPVMQKKTCGWEPSTETVIPRDGGLLLRDTPALMLADGGHLSCFMETTYKSKKEVKLPELHFHHLRMEKLNISDDWKTVEQHESVVASYSQVPSKLGHN

SEQ ID NO: 61 - protein, 225 aa, Montastraea cavernosa
MSVIKPIMEIKLRMQGVVNGHKFVIKGEGEGKPFEGTQTINLTVKEGAPLPFAYDILTSAFQYGNRVFTKYPDDIPDYFKQTFPEGYSWERIMAYEDQSICTATSDIKMEGDCFIYEIQFHGVNFPPNGPVMQKKTLKWEPSTEKMYVRDGVLKGDVNMALLLEGGGHYRCDFRSTYKAKKRVQLPDYHFVDHRIEILSHDNDYNTVKLSEDAEARYSMLPSQAK

SEQ ID NO: 62 - protein, 227 aa, Montastraea faveolata
MSVIKPDMKIKLRMEGAVNGHKFVIEGDGKGKPFEGTQSMDLTVKEGAPLPFAYDILTTVFDYGNRVFAKYPQDIPDYFKQTFPEGYSWERSMTYEDQGICVATNDITLMKGVDDCFVYKIRFDGVNFPANGPVMQKKTLKWEPSTEKMYVRDGVLKGDVNMALLLEGGGHYRCDFKTTYKAKKFVQLPDYHFVDHRIEILSHDKDYNKVKLYEHAEAHSGLPRQAK

SEQ ID NO: 63 - protein, 266 aa, Clavularia sp.
MKCKFVFCLSFLVLAITNANIFLRNEADLEEKTLRIPKALTTMGVIKPDMKIKLKMEGNVNGHAFVIEGEGEGKPYDGTHTLNLEVKEGAPLPFSYDILSNAFQYGNRALTKYPDDIADYFKQSFPEGYSWERTMTFEDKGIVKVKSDISMEEDSFIYEIRFDGMNFPPNGPVMQKKTLKWEPSTEIMYVRDGVLVGDISHSLLLEGGGHYRCDFKSIYKAKKVVKLPDYHFVDHRIEILNHDKDYNKVTLYENAVARYSLLPSQA

SEQ ID NO: 64 - protein, 232 aa, Discosoma striata
MSCSKSVIKEEMLIDLHLEGTFNGHYFEIKGKGKGQPNEGTNTVTLEVTKGGPLPFGWHILCPQFQYGNKAFVHHPDNIHDYLKLSFPEGYTWERSMHFEDGGLCCITNDISLTGNCFYYDIKFTGLNFPPNGPVVQKKTTGWEPSTERLYPRDGVLIGDIHHALTVEGGGHYACDIKTVYRAKKAALKMPGYHYVDTKLVIWNNDKEFMKVEEHEIAVARHHPFYEPKKDK

SEQ ID NO: 65 - protein, 238 aa, Ptilosarcus sp.
MNRNVLKNTGLKEIMSAKASVEGIVNNHVFSMEGFGKGNVLFGNQLMQIRVTKGGPLPFAFDIVSIAFQYGNRTFTKYPDDIADYFVQSFPAGFFYERNLRFEDGAIVDIRSDISLEDDKFHYKVEYRGNGFPSNGPVMQKAILGMEPSFEVVYMNSGVLVGEVDLVYKLESGNYYSCHMKTFYRSKGGVKEFPEYHFIHHRLEKTYVEEGSFVEQHETAIAQLTTIGKPLGSLHEWV

SEQ ID NO: 66 - protein, 231 aa, Zoanthus sp.
MAHSKHGLKEEMTMKYHMEGCVNGHKFVITGEGIGYPFKGKQTINLCVIEGGPLPFSEDILSAGFKYGDRIFTEYPQDIVDYFKNSCPAGYTWGRSFLFEDGAVCICNVDITVSVKENCIYHKSIFNGMNFPADGPVMKKMTTNWEASCEKIMPVPKQGILKGDVSMYLLLKDGGRYRCQFDTVYKAKSVPSKMPEWHFIQHKLLREDRSDAKNQKWQLTEHAIAFPSALA

SEQ ID NO: 67 - protein, 229 aa, Anemonia majano
MALSNKFIGDDMKMTYHMDGCVNGHYFTVKGEGNGKPYEGTQTSTFKVTMANGGPLAFSFDILSTVFKYGNRCFTAYPTSMPDYFKQAFPDGMSYERTFTYEDGGVATASWEISLKGNCFEHKSTFHGVNFPADGPVMAKKTTGWDPSFEKMTVCDGILKGDVTAFLMLQGGGNYRCQFHTSYKTKKPVTMPPNHVVEHRIARTDLDKGGNSVQLTEHAVAHITSVVPF

SEQ ID NO: 68 - protein, 238 aa, Aequorea macrodactyla
MSKGEELFTGIVPVLIELDGDVHGHKFSVRGEGEGDADYGKLEIKFICTTGKLPVPWPTLVTTFSYGIQCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSTQTAISKDRNETRDHMVFLEFFSACGHTHGMDELYK

SEQ ID NO: 69 - protein, 9 aa, Artificial Sequence
  Expressed NtermEGFP fragment (residues 64-72 only)
LTYGVQCFS

SEQ ID NO: 70 - protein, 9 aa, Artificial Sequence
  Expressed NtermEYFP fragment (residues 64-72 only)
FGYGLQCFA

SEQ ID NO: 71 - protein, 9 aa, Artificial Sequence
  Expressed NtermE(F64L)YFP fragment (residues 64-72 only)
LGYGLQCFA